(19) United States
(12) Patent Application Publication
(10) Pub. No.: US 2026/0158283 A1
(43) Pub. Date: Jun. 11, 2026

(54) IMPLANTABLE ELECTRODE ARRAY POSITION MONITORING

(71) Applicant: Cochlear Limited, Macquarie University, NSW (AU)

(72) Inventors: Christopher BENNETT, Macquarie Park, NSW (AU); Nicholas Charles Kendall PAWSEY, North Ryde, NSW (AU); Zachary Mark SMITH, Pymble, NSW (AU); Frank RISI, Newtown, NSW (AU); Paul Michael CARTER, Galson, NSW (AU); Ryan Orin MELMAN, South Coogee, NSW (AU); Peter GIBSON, South Coogee, NSW (AU)

(21) Appl. No.: 18/707,008

(22) PCT Filed: Oct. 31, 2022
(86) PCT No.: PCT/IB2022/060473

§ 371 Date: May 2, 2024

Related U.S. Application Data
(60) Provisional application No. 63/277,253, filed on Nov. 9, 2021.

(51) Int. Cl. A61N 1/372; A61N 1/05; A61N 1/36

(52) U.S. Cl. CPC A61N 1/372; A61N 1/0541; A61N 1/36038

(57) ABSTRACT

Presented herein are techniques to determine the placement/position of one or more parts of an electrode array during or after surgery. The techniques presented herein use a “positioning model” (positioning algorithm) to estimate (predict) the positioning/placement of an electrode array and/or estimate positioning/placement features (e.g., depth of basal electrode, modiolar proximity, polar or Cartesian coordinates of electrodes, etc.) of the electrode array inside of a recipient's body chamber (e.g., cochlea) during and after insertion. More specifically, the techniques presented herein use measurements (e.g., voltage measurements) obtained from the body chamber of the recipient as an input to a trained model/algorithm to estimate/predict the position of an electrode array within the body chamber of the recipient.

### BACKGROUND

### Field of the Invention

The present invention relates generally to monitoring the position of an electrode array during implantation into a recipient.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: determining an insertion depth of a basal part of an elongate electrode array within a body chamber of a recipient, wherein the electrode array comprises a plurality of longitudinally spaced electrodes; obtaining one or more electrical measurements from the body chamber of the recipient; and determining a value for a key position of the elongate electrode array using a relationship between at least the determined insertion depth of the basal part of the elongate electrode array and the one or more electrical measurements.

In another aspect, a method is provided. The method comprises: determining an insertion depth of a basal part of an elongate electrode array within a cochlea of a recipient, wherein the electrode array comprises a plurality of longitudinally spaced electrodes; obtaining a positioning model capturing the mechanical behavior of the elongate electrode array during insertion with respect to at least one of a lateral wall of the cochlea, a modiolar wall of the cochlea, or a mid-modiolar axis of the cochlea; and using the insertion depth of a basal part of an elongate electrode array within the positioning model to determine an intra-surgical electrode location of one or more parts of the elongate electrode array.

In another aspect, a method is provided. The method comprises: inserting an elongate electrode array into a cochlea of a recipient; during a first time period of the insertion, monitoring a position of one or more parts of the elongate electrode array with a first positioning model; and during a second time period of the insertion, monitoring a position of one or more parts of the elongate electrode array with a second positioning model.

In another aspect, one or more non-transitory computer readable storage media are provided. The one or more non-transitory computer readable storage media comprise instructions that, when executed by a processor, cause the processor to: determine an insertion depth of a basal part of an elongate electrode array within a body chamber of a recipient; receive one or more electrical measurements obtained from the body chamber of the recipient; and estimate a position of the elongate electrode array using a statistical or probabilistic relationship between at least the determined insertion depth of the basal part of the elongate electrode array and the one or more electrical measurements.

In another aspect, a one or more non-transitory computer readable storage media are provided. The one or more non-transitory computer readable storage media comprise instructions that, when executed by a processor, cause the processor to: receive one or more electrical measurements obtained from the cochlea of the recipient via a stiffening sheath inserted into the cochlea; and use a positioning model to estimate a position of the stiffening sheath within the cochlea, wherein the positioning model is mathematically described by at least one of a statistical model or a probabilistic model.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is a high-level flowchart of a method, in accordance with certain embodiments presented herein;

FIG. 3A is an annotated medical image illustrating a basal depth of insertion of an electrode array, in accordance with embodiments presented herein;

FIG. 3B is an annotated medical image illustrating modiolar proximity of an electrode array, in accordance with embodiments presented herein;

FIG. 3C is an annotated medical image illustrating angular depth of insertion of an electrode array, in accordance with embodiments presented herein;

FIG. 4 is a table illustrating aspects of example positioning models, in accordance with certain embodiments presented herein;

FIG. 5A is a schematic diagram illustrating further details of an example positioning model using a probabilistic approach to determine positions of one or more parts of an electrode array, in accordance with certain embodiments presented herein;

FIG. 5B is an annotated medical image illustrating positions of electrodes of an electrode array, in accordance with certain embodiments presented herein;

FIG. 6 is a graph illustrating a distribution of the estimates/predictions generated by a probabilistic positioning model, in accordance with certain embodiments presented herein;

FIG. 7 is a flowchart of an example method associated with use of a positioning model for electrode array position estimation, in accordance with certain embodiments presented herein;

FIG. 8 is a flowchart of an example method associated with use of a positioning model for modiolar proximity estimation, in accordance with certain embodiments presented herein;

FIG. 9 is a graph illustrating a number of estimates generated using an example positioning model, for several insertions, in accordance with certain embodiments presented herein;

FIG. 10 is a schematic diagram illustrating further details of a probabilistic positioning model, in accordance with certain embodiments presented herein;

FIGS. 11A, 11B, 11C, 11D, and 11E are a series of images illustrating different stages of an optimal insertion of an electrode array into a plastic model of a cochlea;

FIG. 12 is an annotated image illustrating a potential problem that can occur during insertion of an electrode array into a plastic model of a cochlea;

FIG. 13 is a graph illustrating including curves indicating of the distance of electrodes of the electrode array of FIG. 12 relative to the modiolus as the insertion progresses and the tip becomes stuck;

FIG. 14 is a diagram illustrating an electrode array having lateral electrodes disposed therein, in accordance with certain embodiments presented herein;

FIGS. 15A, 15B, and 15C are images illustrating optimal positioning of a sheath during insertion of an electrode array into a plastic model of a cochlea;

FIG. 16 is an annotated image illustrating sub-optimal positioning of a sheath during insertion of an electrode array into a plastic model of a cochlea;

FIGS. 17A and 17B are schematic diagrams illustrating a sheath with electrodes disposed thereon, in accordance with certain embodiments presented herein;

FIG. 18 is a schematic diagrams illustrating a sheath with electrodes disposed thereon, where the sheath is in a sub-optimal position for insertion of an electrode array;

FIG. 19A is a schematic diagram illustrating a sheath with electrodes disposed thereon during a round window insertion, where the sheath is improperly angled for insertion of an electrode array;

FIG. 19B is a schematic diagram illustrating a sheath with electrodes disposed thereon during a round window insertion, where the sheath is correctly angled for insertion of an electrode array;

FIG. 19C is another schematic diagram illustrating a sheath with electrodes disposed thereon during a round window insertion, where the sheath is improperly angled for insertion of an electrode array;

FIG. 19D is a graph illustrating distance from modiolus versus angle of sheath for insertion shown in FIGS. 19A, 19B, and 19C;

FIG. 20A is a schematic diagram illustrating a sheath with electrodes disposed thereon during a cochleostomy insertion, where the sheath is improperly angled for insertion of an electrode array;

FIG. 20B is a schematic diagram illustrating a sheath with electrodes disposed thereon during a cochleostomy insertion, where the sheath is correctly angled for insertion of an electrode array;

FIG. 20C is another schematic diagram illustrating a sheath with electrodes disposed thereon during a cochleostomy insertion, where the sheath is improperly angled for insertion of an electrode array;

FIG. 20D is a graph illustrating distance from modiolus versus angle of sheath for insertion shown in FIGS. 20A, 20B, and 20C;

FIG. 21A is a schematic diagram illustrating a straight electrode array inserted through the round window;

FIG. 21B is a schematic diagram illustrating a straight electrode array inserted through a cochleostomy;

FIGS. 22A, 22B, 22C, 22D, and 22E are a series of images illustrating the trajectory of an electrode array during insertion into a plastic cochlea model;

FIGS. 23A and 23B are schematic diagrams illustrating an example in which an electrode array begins to buckle in the basal section as the tip of the electrode array lodges in the tissue of the lateral wall;

FIGS. 24A and 24B are schematic diagrams illustrating further details of an example in which an electrode array begins to buckle during insertion;

FIGS. 25A and 25B are schematic diagrams illustrating an example in an electrode array begins to buckle in an apical section during insertion;

FIGS. 26A and 26B are schematic diagrams illustrating an example where an electrode array begins to buckle in multiple locations during insertion;

FIGS. 27A and 27B are schematic diagrams illustrating one example technique to visually display a degree certainty or likely accuracy of an estimated position of an electrode array within a cochlea, as determined in accordance with certain embodiments presented herein;

FIG. 28 is a flowchart of an example method, in accordance with certain embodiments presented herein;

FIG. 29 is a flowchart of another example method, in accordance with certain embodiments presented herein;

FIG. 30 is a flowchart of another example method, in accordance with certain embodiments presented herein;

FIG. 31 is a schematic diagram illustrating a retinal prosthesis system with which aspects of the techniques presented herein can be implemented; and

FIG. 32 is a block diagram illustrating a computing device/system configured to implement certain embodiments presented herein.

### DETAILED DESCRIPTION

A growing number of implantable medical devices include an electrode array that is configured to be implanted within a recipient. For example, cochlear implants typically include an electrode array configured to be implanted within the cochlea (e.g., scala tympani) of a recipient. The position of the electrode array within the cochlea, in particular the proximity of the electrode array to the modiolus, can impact the performance of the cochlear implant. As such, it is desirable for a surgeon to know the position of the electrode array during surgery so that they can optimize the position thereof.

Currently, the only means of dependably determining the position of an electrode array is via imaging. While the use of imaging (e.g., Fluoroscopy) is possible during surgery, such intra-operative imaging is cumbersome, time-consuming, expensive, and adds x-ray exposure for the recipient and operating staff. It is reasonably convenient to take post-operative x-rays or CT scans, but this still adds x-ray exposure, cost, and can only detect the position after the surgery is completed. Therefore, there is a need for a way to determine the electrode array position in a recipient, such as in the cochlea, by means other than taking imaging. Furthermore, it is desirable to be able to make a measurement of the location of the electrode array in the course of an operation and, preferably, multiple times so that the position of the electrode array can be optimized and/or to ensure no issues arise (e.g., detect tip fold-over, detect outward arching and pressing on the lateral wall which could cause undesirable pressure on lateral wall tissues and possibly loss of residual hearing, evaluate final positioning at some distance from the modiolus rather than in close proximity, etc.). That is, there is a need to provide surgeons with the ability to monitor the dynamic of the electrode array during insertion and provide feedback so the surgeon can take corrective action to correct the situation should a problem arise (e.g., should the tip become caught). It is also desirable to be able to make a measurement of an electrode array post-operatively so that the position of the electrode array can be confirmed without x-ray exposure.

In view of the above, presented herein are techniques to determine the placement/position of one or more parts of an electrode array during or after surgery without intra-operative use of imaging, such as x-rays, CT, or fluoroscopy. The techniques presented herein use a “positioning model” (positioning algorithm) to estimate (predict) the positioning/placement of an electrode array and/or estimate positioning/placement features (e.g., depth of basal electrode, modiolar proximity, polar or Cartesian coordinates of electrodes, etc.) of the electrode array inside of a recipient's body chamber/cavity (e.g., cochlea) during and after insertion. More specifically, the techniques presented herein use measurements (e.g., voltage measurements) obtained from the body chamber of the recipient as an input to a trained model/algorithm to estimate/predict the position of an electrode array within the body chamber of the recipient

As described further below, the estimation of the position of one or more parts of an electrode array can include, for example, modiolar proximity (e.g., position of the electrode array with respect the modiolus) using a statistical positioning model capturing the relationship between electrical measurements (and derived features) and modiolar proximity. The estimation of the position of one or more parts of an electrode array can also include electrode array positioning/placement (e.g., polar and/or Cartesian coordinates of the electrodes, from which modiolar proximity can be derived thereafter. The electrode array positioning can be determined using, for example, using a statistical positioning model (e.g., principle component regression) capturing the relationship between electrical measurements (and derived features) and basal electrode depth of insertion; using a statistical positioning model (e.g., principle component regression) capturing the relationship between electrical measurements (and derived features) and mean middle electrodes modiolar proximity; using spiral models to establish the modiolus and lateral wall spirals; using a probabilistic (e.g., Monte Carlo) positioning model (simulation) to capture the physical/mechanical properties of electrode array and cochlea such to estimate the placement. In the probabilistic positioning model, the depth of insertion is the starting position, the mean middle proximity model establishes a region of likelihood to filter out poor estimates, and the modiolus spiral model and the lateral spiral model establish physical boundary conditions to filter out poor estimates.

Merely for ease of description, the techniques presented herein are primarily described with reference to implantation of stimulating assemblies associated with a specific implantable medical device system, namely a cochlear implant system, into the cochlea of a recipient. However, it is to be appreciated that the techniques presented herein may also be used to implant other types of stimulating assemblies into other body chambers/cavities and/or stimulating assemblies associated with other types of implantable medical devices. For example, the techniques presented herein may be implemented with other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein may also be implemented by dedicated tinnitus therapy devices and tinnitus therapy device systems. In further embodiments, the presented herein may also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

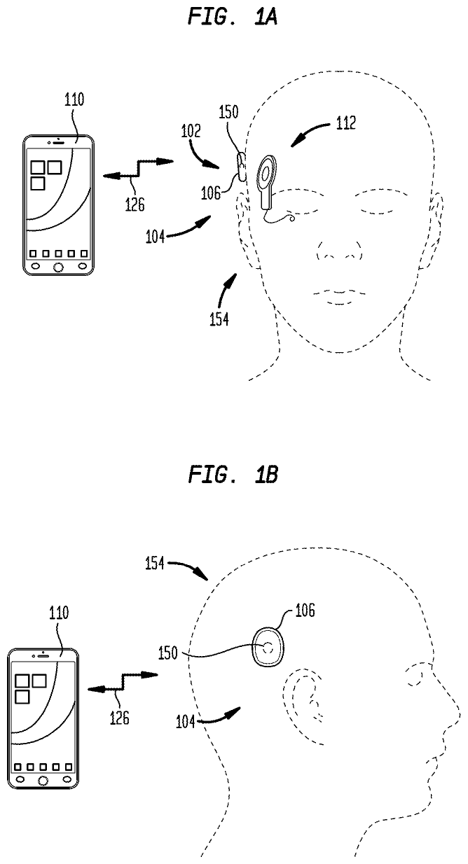

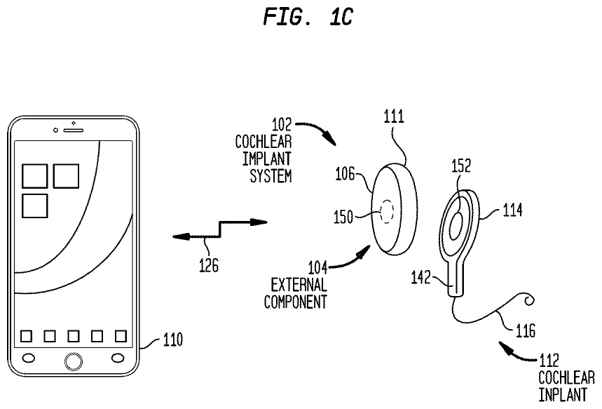

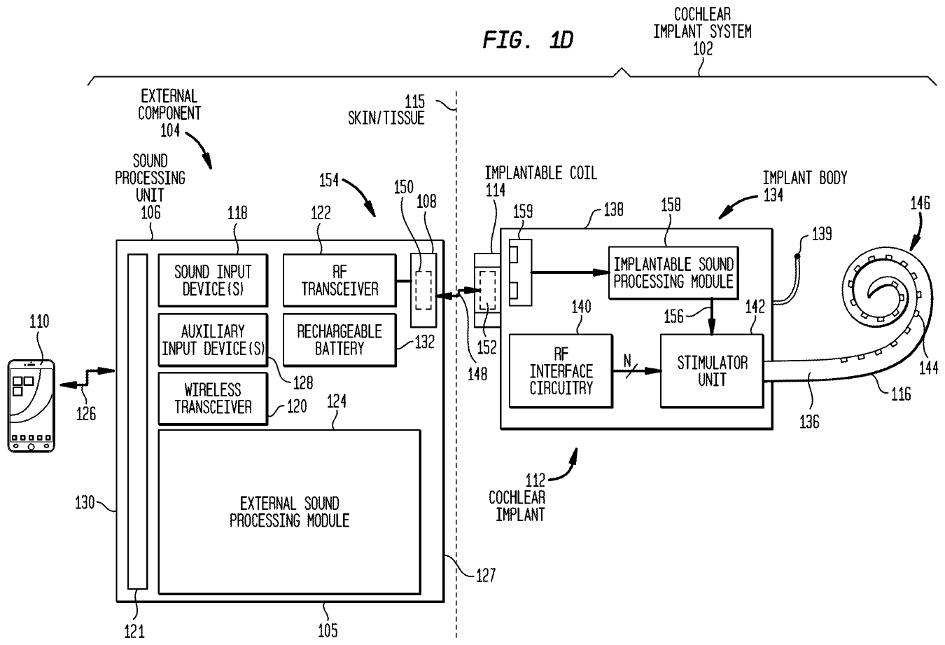

FIGS. 1A-1D illustrates an example cochlear implant system 102 with which aspects of the techniques presented herein can be implemented. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an implantable coil 114, an implant body 134, and an elongate electrode array 116 configured to be implanted in the recipient's cochlea.

In the example of FIGS. 1A-1D, the sound processing unit 106 is an off-the-ear (OTE) sound processing unit, sometimes referred to herein as an OTE component, which is configured to send data and power to the implantable component 112. In general, an OTE sound processing unit is a component having a generally cylindrically shaped housing 111 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component may comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110 is a computing device, such as a computer (e.g., laptop, desktop, tablet), a mobile phone, remote control unit, etc. As described further below, the external device 110 comprises a telephone enhancement module that, as described further below, is configured to implement aspects of the auditory rehabilitation techniques presented herein for independent telephone usage. The external device 110 and the cochlear implant system 102 (e.g., OTE sound processing unit 106 or the cochlear implant 112) wirelessly communicate via a bi-directional communication link 126. The bi-directional communication link 126 may comprise, for example, a short-range communication, such as Bluetooth link, Bluetooth Low Energy (BLE) link, a proprietary link, etc.

Returning to the example of FIGS. 1A-1D, the OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external device 110). However, it is to be appreciated that one or more input devices may include additional types of input devices and/or less input devices (e.g., the wireless short range radio transceiver 120 and/or one or more auxiliary input devices 128 could be omitted).

The OTE sound processing unit 106 also comprises the external coil 108, a charging coil 130, a closely-coupled transmitter/receiver (RF transceiver) 122, sometimes referred to as or radio-frequency (RF) transceiver 122, at least one rechargeable battery 132, and an external sound processing module 124. The external sound processing module 124 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intracochlear electrode array 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, electrode array 116 is configured to be at least partially implanted in the recipient's cochlea. Electrode array 116 includes a plurality of longitudinally spaced intracochlear electrical stimulating contacts (electrodes) 144 that collectively form a contact array 146 for delivery of electrical stimulation (current) to the recipient's cochlea.

Electrode array 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive, and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 1D, the cochlear implant 112 includes a plurality of implantable sound sensors 159 and an implantable sound processing module 158. Similar to the external sound processing module 124, the implantable sound processing module 158 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 159 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 159) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 159 in generating stimulation signals for delivery to the recipient.

As noted, during the insertion of an intra-cochlear electrode array, the surgeon has minimal feedback of the placement of the electrode array inside the cochlea (e.g., the insertion is performed “blind”). The physical placement or position of the electrode array inside of the cochlea can only be confirmed using intraoperative or postoperative imaging. For example, an x-ray can confirm whether the placement of an electrode array conforms to the general curvature of the spiral of the cochlea, but cannot provide information about the distance between electrode contacts (electrodes) and the modiolus (e.g., modiolar proximity). High quality CT imaging can be used to derive information such as depth of insertion and modiolar proximity, and thus can confirm the quality of placement of the electrode array. However, not all surgical theatres have the equipment to conduct intraoperative imaging, and many don't incorporate CT imaging as part of their postoperative clinical routines. Further, CT imaging poses a risk to patients due to exposure to radiation.

A cochlear implant system is able to provide different current stimulation paradigms across one or many of the electrodes of the electrode array. The electrodes of the electrode array and extracochlear electrodes can also be used to make electrical measurements (e.g., potential difference (voltage) and/or impedance (e.g., resistance, reactance, and impedance)).

With all other variables being equal, the changing of the placement of the electrode array inside of the cochlea will change the nature of the electrical measurements. For example, the cross-sectional area of the fluid-filled cochlea duct decreases as the angular depth increases, appealing to the resistance equation

$$( R = ρ \frac{L}{A} ;$$

where R is resistance, ρ is resistivity, L is the length and A is the cross-sectional area), the resistance at a location deeper in the cochlea would be greater than a location shallower in the cochlea. Further, as an electrode contact is closer to a structure of the cochlea, the effective cross-sectional area decreases, thus increasing measured resistance.

Presented herein are techniques that use a plurality of intraoperatively collected electrical measurements, such as impedance measurements (e.g., CG, MP1, MP2 and MP12), transimpedance matrices (TIMs), and intracochlear bipolar impedances, in combination with intraoperative CT imaging or postoperative CT imaging to identify relationships of electrical measurements and/or features thereof and coordinates of the placement of electrodes inside of the cochlea. The techniques presented use the relationships of electrical measurements and/or features thereof and coordinates of the placement of electrodes inside of the cochlea to produce models and/or algorithms (e.g., statistical models, probabilistic models, first principle models, etc.) to estimate the real-time position of the electrodes of an electrode array inside of the cochlea of a recipient, during and/or after implantation/insertion. During surgery and/or after the implantation of the electrode array, electrical measurements can be taken, processed, and input into the models and/or algorithm to estimates of the position of the electrodes. As used herein, the position of the electrodes can include the position relative to the modiolus (e.g., modiolar proximity), angle of insertion, insert distance from the mid-modiolar axis, or other information relating to the placement or position of one or more parts of an electrode array.

In view of the above, the techniques presented herein avoid the need for intraoperative or postoperative imaging, and, in turn, reduce patient exposure to radiation from imaging, reduce costs, etc. The techniques presented herein also provide useful feedback that can be used to optimize the electrode array placement to maximize outcomes, and to provide confidence for the surgeon to know that the electrode array is in the best place prior to finishing surgery. This is particularly beneficial for perimodiolar electrodes where the optimal position is close to the modiolus.

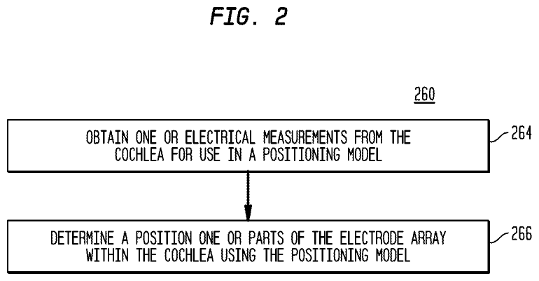

FIG. 2 is a high-level flowchart of an example method 260, in accordance with certain embodiments presented herein. Method 260 begins at 262 where a system obtains (e.g., receives, determines, etc.) electrical measurements from the cochlea for use in “positioning model.” The electrical measurements are obtained via (using) at least a portion of the electrode array inserted into the cochlea. At 264, the system determines a position or placement of one or more parts of the electrode array within the cochlea using the one or more electrical measurements as input to the positioning model. As used herein, the positioning model incorporates (accounts for) mechanical properties in order to predict or estimate for example, modiolar proximity of one or more parts of the electrode array (e.g., modiolar proximity of the basal, apical, or middle parts of the electrode array), depth of insertion of one or more electrodes, such as the most basal electrode, angular depth of insertion, position of one or more of the electrodes, etc. The operations performed at 262 and 264, which vary in accordance with different embodiments presented herein, are described in further detail below.

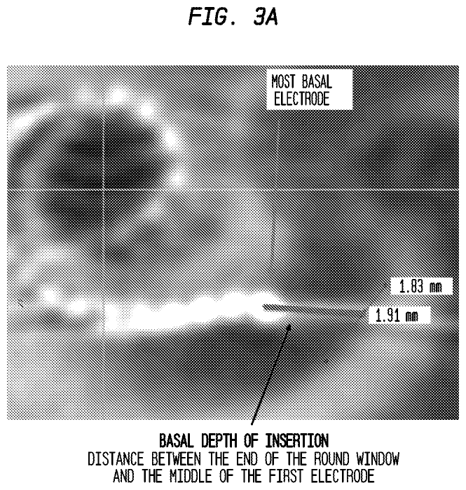

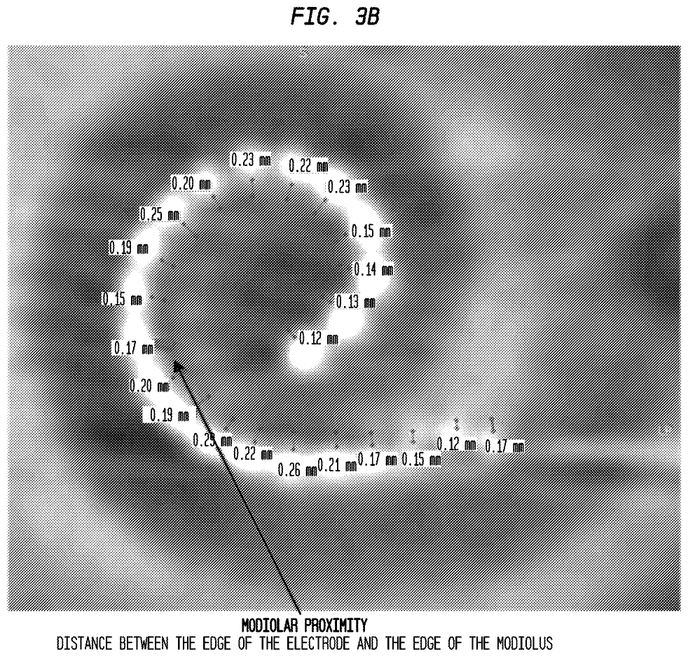

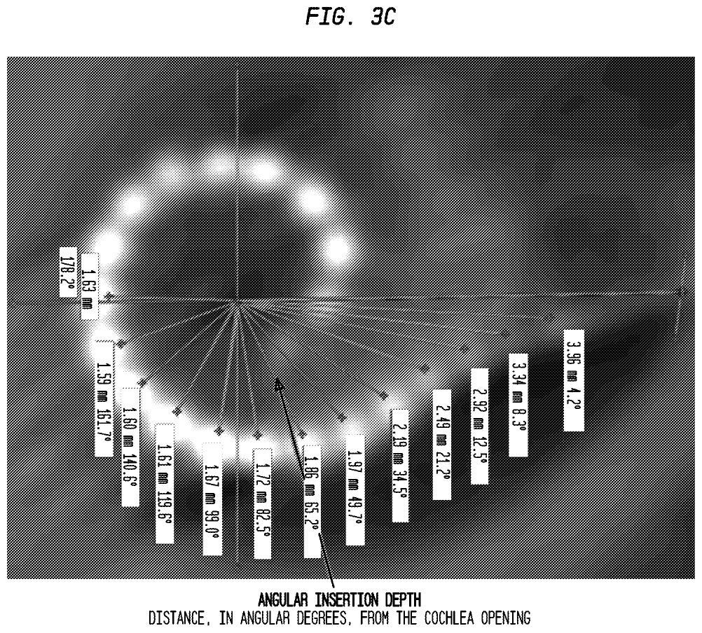

FIGS. 3A, 3B, and 3C are annotated medical images (e.g., CT scans) of an electrode array inserted into a cochlea. FIG. 3A schematically illustrates the insertion depth of a basal part of an electrode array at least partially inserted into a cochlea of a recipient as, in this example, a distance between the most basal (last inserted) electrode and the cochlea opening (e.g., round window, cochleostomy, etc.). FIG. 3B schematically illustrates an example position of a part of the electrode array within the cochlea, namely the modiolar proximity of the electrodes. As used herein, the modiolar proximity is a distance between the edge of an electrode and an edge of the cochlea modiolus. The modiolar proximity can be determined for individual electrodes or groups of electrodes (e.g., basal electrodes, middle electrodes, apical electrodes, etc.).

FIG. 3C schematically illustrates another example position of a part of the electrode array within the cochlea, namely the angular insertion depth of the electrodes. As used herein, the angular insertion depth is the distance of an electrode or other part of the electrode array from the cochlea opening, measured/reported as an angle (degrees). Examples of other positions of one or more parts of an electrode array include, but are not limited to, a distance between one or more of the electrode and a lateral wall of the cochlea, polar or Cartesian coordinates of one or more of the electrodes, a position of a non-basal area of the electrode array, a position of a point near middle of the elongate electrode array, etc.

As noted, the modiolar proximity (FIG. 3B), angular insertion depth (FIG. 3C), and/or other placements/positions of one or more parts of an electrode array are determined through use of a “positioning model.” That is, in accordance with certain embodiments presented herein, one or more input parameters are obtained and input into a predetermined model that is generated based on normative data associated with (obtained from) insertions of stimulating assemblies in other recipients. The positioning model is used to determine, based the input parameters, the real-time position of one or more parts (e.g., electrodes) of the electrode array inserted into the particular recipient.

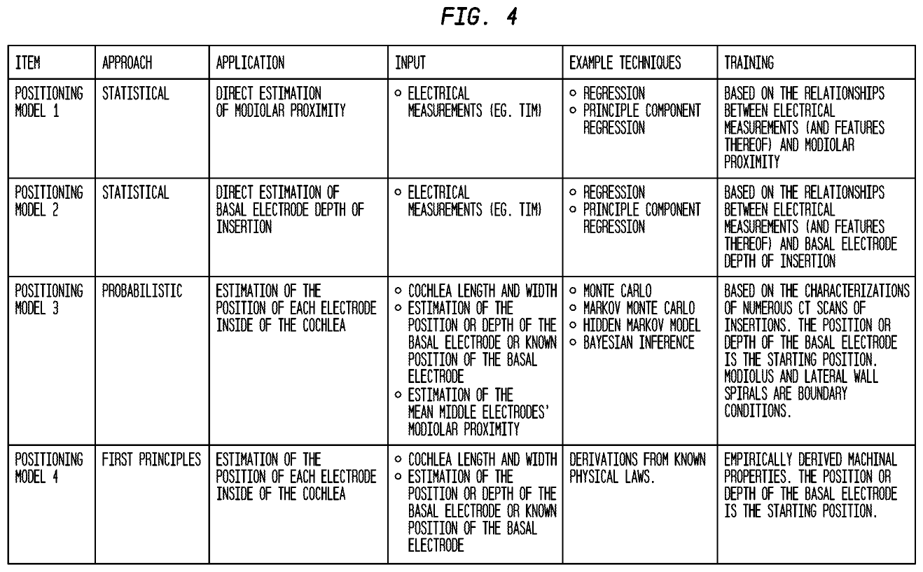

Positioning models presented herein can have a number of different properties and forms in various embodiments. FIG. 4 is a table summarizing four (4) example positioning models that can be used in different embodiments presented herein. In the table shown in FIG. 4, the four example models are referred to as: positioning model 1, positioning model 2, positioning model 3, and positioning model 4.

Positioning models 1 and 2 are each statistical models that can be applied to, for example, directly estimate modiolar proximity and/or directly estimate a depth of insertion of the most basal electrode. Positioning models 1 and 2 operate based on one or more electrical measurements obtained via the electrode array while located in the cochlea, such as transimpedance measurements. Positioning models 1 and 2 can use, for example, regression or principle component regression techniques and are trained based on the relationships between electrical measurements (and features thereof) and modiolar proximity obtained from normative data (e.g., data from electrode array insertions in other recipients).

In the example of FIG. 4, positioning model 3 is a probabilistic model that can be applied to, for example, estimate a position or angular insertion of one or more electrodes within the cochlea, determine modiolar proximity from the electrode positions, etc. Positioning model 3 operates based on the cochlea dimensions (e.g., length and width, determined from pre-operative imaging), an estimate of position or insertion depth of the basal electrode (e.g., determined from electrode array position model 2 and/or from electrical measurements), and/or estimate of the mean modiolar proximity (e.g., as determined from electrode array position model 1 and/or electrical measurements). Positioning model 3 can use, for example, Monte Carlo techniques, Markov Monte Carlo techniques, Hidden Markov Model techniques, Bayesian interference techniques, and/or other techniques and is trained based on the based on characterizations of medical imaging results (CT scans) of electrode array insertions in other recipients. In this example, the position or depth of the basal electrode is the starting point for the estimation and modiolus and lateral wall spirals (as determined from the cochlea dimensions) are boundary conditions.

In the example of FIG. 4, positioning model 4 is a first principles mechanical model that can be applied to, for example, estimate a position or angular insertion of one or more electrodes within the cochlea, determine modiolar proximity from the electrode positions, etc. Positioning model 4 operates based on the cochlea dimensions (e.g., length and width, determined from pre-operative imaging) and an estimate of position or insertion depth of the basal electrode. Positioning model 4 is derived from physical laws and the mechanical properties of the electrode array and is trained based on the based on empirically derived machinal properties. Similar to positioning model 3, the position or depth of the basal electrode is the starting point for the estimation.

It is to be appreciated that the four specific positioning models shown in FIG. 4 are merely illustrative and that other models can be used in certain embodiments presented herein. It is also to be appreciated that the example models are not mutually exclusive and could be combined with one another in different manners. For example, a further example model could use a probabilistic approach (e.g., aspects of positioning model 3) in combination with a first principles approach (e.g., aspects of positioning model 4). It is also be appreciated that different models could be used at different times during a same surgical procedure. For example, one such example could use positioning model 4 during a first portion/part of a surgical procedure, but switch to use of positioning model 3 during a second portion/part of the surgical procedure.

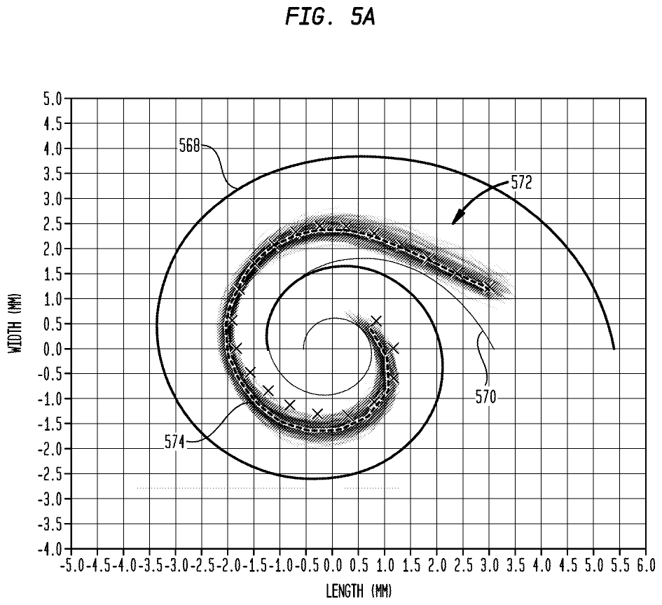

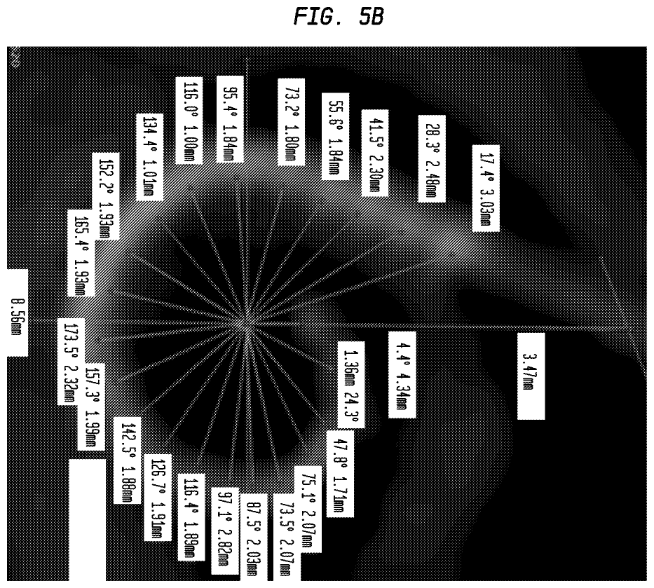

FIG. 5A is a diagram illustrating further details of positioning model 3 using a probabilistic approach to determine positions of one or more parts of an electrode array from a determined insertion depth of the basal part of the elongate electrode array and the one or more electrical measurements. In FIG. 5A, the “X” marks indicate the positions of the electrodes taken from the corresponding CT scans, shown in FIG. 5B. The spiral 568 is the estimated lateral wall, as determined from cochlea dimensions referred to as the “A” and “B” dimensions. The “A” dimension of the cochlea is the length of the tangent from the cochlea opening, in this example the round window, to the lateral wall passing through the mid-modiolar axis (e.g., cochlea length). The “B” dimension of the cochlea as the length of the tangent passing through the mid-modiolar axis, perpendicular to the A dimension tangent, between each side of the lateral wall (e.g., cochlea width). The A and B dimensions are also used to estimate the modiolus wall, shown as spiral 570 in FIG. 5A.

Shown also in FIG. 5A are a plurality of lines 572 that each represent an output of a single estimate generated by the probabilistic algorithm (e.g., each representing a single Monte Carlo run/iteration trained using observed physical positions of electrodes, given basal electrode depth/position). Line 574 represents the average of the probabilistic algorithm outputs. In FIG. 5A, the estimates of the depth of the basal electrode and region of likelihood formed by the estimate of the mean middle electrode modiolar proximity are not displayed in the image.

In this example, the estimate of the position of the basal electrode is determined from electrical measurements, such as the transimpedance measurements (e.g., positioning model 2). Alternatively, the estimate of the position of the basal electrode could be determined based on robotic insertion data (e.g., known depth of the basal electrode given the robotic movement), visual inspection of markers on the electrode array, etc.

In other words, FIG. 5A illustrates an example in which the cochlea dimensions (e.g., A and B dimensions) are used to individualize a lateral wall spiral and a modiolus spiral for the recipient. During surgery, the electrical measurements are run (e.g., transimpedance measurements) to estimate depth of insertion and relative location to the modiolus (modiolar wall). The measurements are also to estimate the mean middle electrode modiolar proximity. The system then runs a probabilistic algorithm (e.g., Monte Carlo simulation) to generate a number of estimates of the position of the electrodes. The average of these estimates provides an accurate estimate of the likely position of the electrodes. FIG. 6 is a graph illustrating a distribution of the estimates/predictions generated by the probabilistic algorithm.

As noted above, certain aspects of the techniques presented herein utilize electrical measurements taken via the electrode array during or after insertion of the electrode array into the cochlea. The electrical measurements that can be taken using the electrode array and cochlear implant system include, but are not limited to, standard impedance measurements, transimpedance measurements, intracochlear bipolar impedance measurements, intracochlear nested bipolar impedances, etc.

Standard impedances can include measurement of a potential difference between a stimulation electrode and a first extracochlear electrode, divided by the stimulation current (MP1), measurement of a potential difference between a stimulation electrode and a second extracochlear electrode, divided by the stimulation current (MP2), measurement of a potential difference between a stimulation electrode and both the first and second extracochlear electrodes, divided by the stimulation current (MP12), measurement of a potential difference between a stimulation electrode and the remainder of the intracochlear electrodes, divided by the stimulation current (CG), etc. The transimpedance measurements can include a three-point measurement were, for each intracochlear electrode, a stimulation electrode is stimulated between the second extracochlear electrode and the potential difference is measured between each intracochlear electrode (recording electrode) and the extracochlear electrode, and where the potential difference measurements are divided by the stimulation current. Alternatively, the transimpedance measurements can include a four-point measurement where, for each intracochlear electrode, a stimulation electrode is stimulated between the first extracochlear electrode and the potential difference is measured between each intracochlear electrode (recording electrode) and the second extracochlear electrode, and where the potential difference measurements are divided by the stimulation current.

Intracochlear bipolar impedance measurements can include measurements where, for different permutations of pairs of intracochlear electrodes, current is stimulated between the electrode pair, potential difference is measured and divided by the stimulation current. Intracochlear nested bipolar impedances can include measurements where, for different permutations of pairs of intracochlear electrodes spaced at least two electrode pads apart, current is stimulated between the electrode pair, an electrode pair situated between the stimulation pair are used to measure potential difference and this is divided by the stimulation current.

The electrical measurements can be processed to produce/extract new features that better correlate to various placement features. Examples of extracted features from TIMs include current flow asymmetry (CF_{asym}, Equation 1) and TIM range (range, Equations 2 and 3).

$$C {F}_{asym , x} = \frac{abs ( {Z}_{x , x - 2} - {Z}_{x , x - 1} )}{abs ( {Z}_{x , x + 2} - {Z}_{x , x + 1} )} , \qquad ( Equation 1 )$$

where the CF_{asym} is calculated for given electrode x, electrode x is the stimulating electrode, the ratio of the absolute differences between the trans-impedances (Z_{x,y}, where x is the stimulating electrode and y is the recording electrode) of the electrode pairs adjacent to the stimulating electrode is calculated.

$${TIM}_{x , x} = NaN , for 1 < x < 22 \qquad ( Equation 2 )$$

$${range}_{x} = \frac{min ( {TIM}_{x} ) - max ( {TIM}_{x} )}{min ( TIM )} \qquad ( Equation 3 )$$

where TIM is the transimpedance matrix, TIMₓ is the set of impedance and transimpedance measurements for stimulating electrode x; TIM_{x,x} is the impedance measurement recorded when stimulating on electrode x; the impedances recorded on the stimulating electrodes are removed; the range is the difference between the maximum and minimum transimpedances of the stimulating electrode divided by the minimum transimpedance value of the TIM.

Other features can be extracted from electrical measurements through the application of dimensionality reductions algorithms such as principle component analysis.

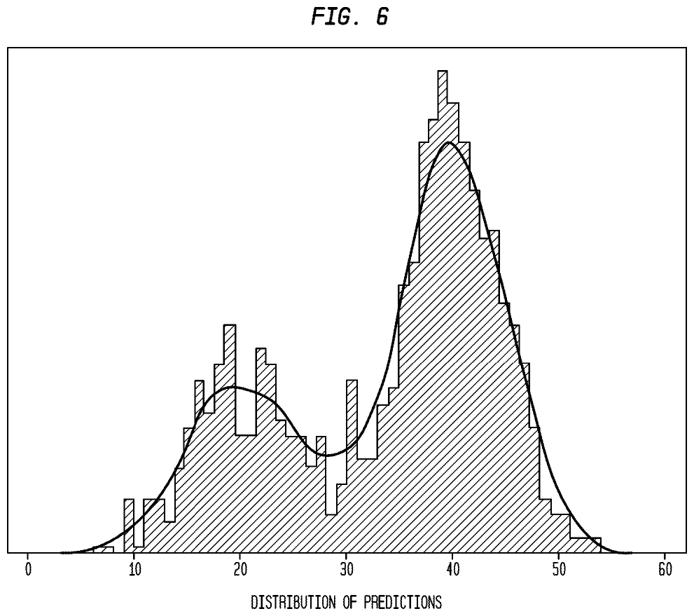

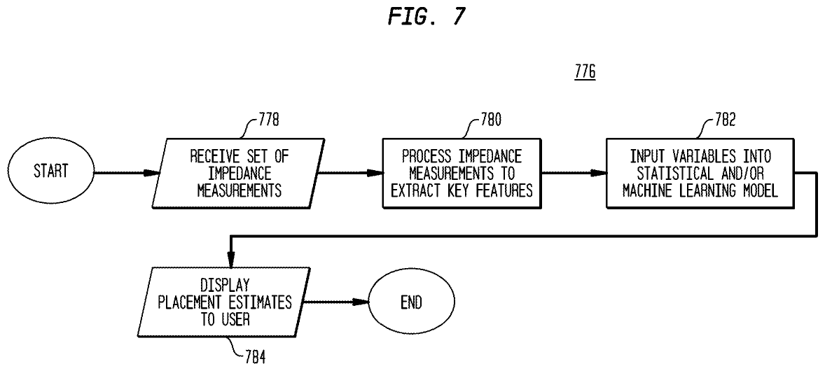

FIG. 7 is a flowchart of an example method 776 associated with use of a positioning model for electrode array position estimation, in accordance with certain embodiments presented herein. At 778, the system obtains or receives a set of impedance measurements and, at 778, the impedance measurements are processed to extract key features. At 780, the input impedances and key features are input into the positioning model (e.g., statistical model, probabilistic model, etc. model) to estimate electrode array positioning. At 782, the estimated positioning is displayed to a user.

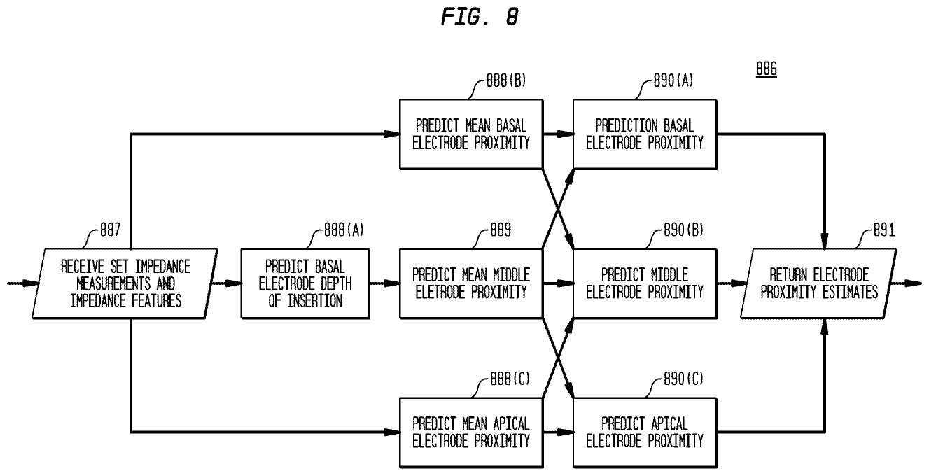

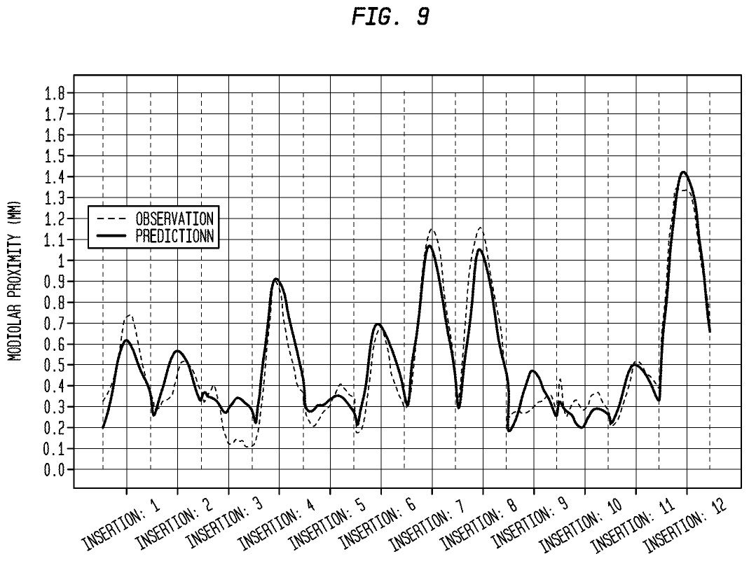

FIG. 8 is a flowchart of an example method 886 associated with use of a positioning model for modiolar proximity estimation, in accordance with certain embodiments presented herein. At 887, the system receives the set of impedance measurements and impedance features (e.g., obtained in FIG. 7). The system uses the measures and key features to estimate the basal electrode depth of insertion at 888(A), to estimate the mean basal electrode proximity at 888(B), and the mean apical electrode proximity at 888(C). A mean middle electrode proximity estimate is performed at 889 based on the estimated basal electrode depth of insertion. As shown, the individual basal electrode estimation models at 890(A) receive mean basal and mean middle electrode estimates as inputs, the individual middle electrode estimation models at 890(B) receive the mean section estimate as inputs, and the individual apical electrode estimation models at 890(C) receive mean apical and mean middle electrode estimates as input. The resultant electrode modiolar proximity estimates are returned at 891. FIG. 9 is a graph demonstrating a range of estimates for several insertions denoted vertical segmented lines.

In certain embodiments, the positioning models described herein can be enhanced using physical properties of the cochlea. In particular, the physical properties of the cochlea influence how the electrode array can be placed inside of the cochlea. With all things being equal, a smaller cochlea will limit the insertion depth and reduce distances from the electrode contacts to the modiolus in comparison to a larger cochlear. This is due to the smaller diameter and cross-sectional area of the scala tympani of the smaller cochlea. It is deduced that the physical properties of the cochlear, measured preoperatively from high quality imaging, can be incorporated as input variables into certain positioning models presented herein to further improve the accuracy. Key features of the physical properties that can be incorporated include, for example, the dimensions (e.g., A and B dimensions) as determined from preoperative imaging scans.

Following the estimation of the distance of the electrodes to the modiolus, the system can perform post-processing to constrain the estimates. For example, using the A and B dimensions annotated from a preoperative CT scan can be used to produce approximations of the cochlea's modiolar and lateral wall spirals. At a given angular depth in the cochlear, the distance between the approximated spirals and considering the thickness of the electrical array gives the maximum modiolar distance at that depth. If the estimate is greater than this estimated maximum, the estimate is reduced to the maximum.

In certain embodiments, the placement of insertion of the electrode array in the cochlea can be estimated by combining electrical measurements and physical models of the cochlea and electrode array. In such examples, the electrode array path is determined from the: (1) estimated depth of insertion of the most basal electrode; (2) the likely position of the most apical electrode; (3) the approximated spirals of the modiolus and lateral walls; (4) the physical dimensions of the electrode array, including length, diameter, spacing of the electrodes, and electrodes length and width; and the insertion approach.

It has been observed that the transimpedance measurements can be used to estimate depth of insertion of the electrode array. The transimpedance measurements can be combined with other electrical measurements such as the standard set of impedances and intracochlear nested bipolar impedances in a positioning model to produce an enhanced depth of insertion estimate.

Many in vivo insertions of stimulating assemblies with annotated post-operative annotated CT scans are used to generate the distribution of modiolar proximities and distribution of depth of insertions. From these distributions, expected values and ranges of likely placement of the electrode array can be derived. The depth and the estimate of the depth of the most basal electrode of the electrode array and the insertion approach give the basal anchorage point of the electrode array. The likely placement of the most placement of the most apical electrode forms the apical anchorage points of the stimulating assemblies. The approximated spirals of the modiolus and the lateral wall form the boundary conditions. Given the known length between the most basal electrode and the most apical electrode a non-linear optimal curve (or path) is derived using an optimization algorithm. At regular intervals (based on the known distances between electrodes) from the most basal electrode to the most apical electrode, electrodes are placed. If insufficient electrodes are placed, the curve is further optimized to ensure that all required electrodes are fitting along the path.

The apical electrode anchorage point is based on a region of high likelihood from derived distributions of known modiolar proximities and depth of insertions. In this region of high likelihood there may be many anchorage points that could be selected. In turn, the electrode pathing algorithm can produce many paths from the apical electrode region of high likelihood of placement to the estimated placement of the basal electrode. Using the electrical measurement inputs, estimates of sectional modiolar proximities can be used to select the path that has the least deviations from the estimates.

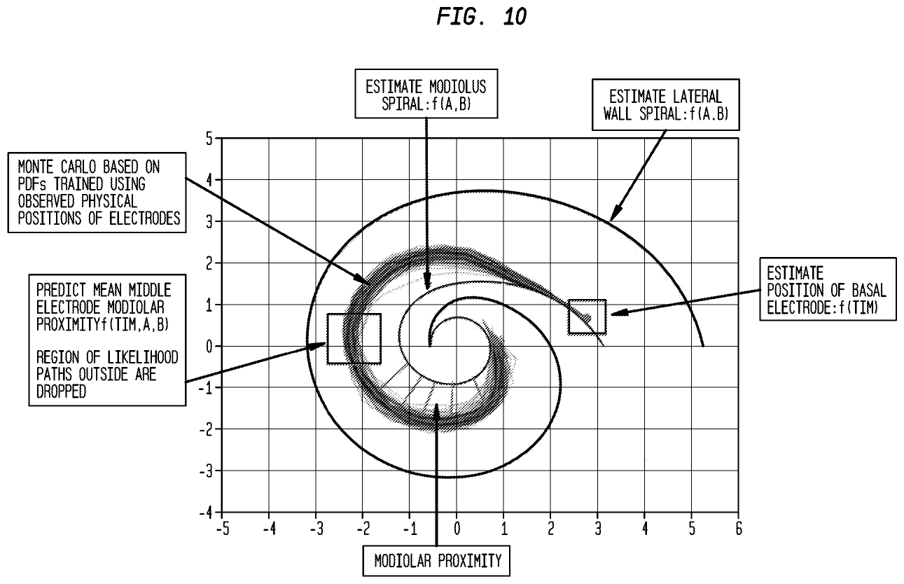

FIG. 10 is a diagram demonstrating one implementation of this technology. Using features of electrical measurements and dimension of a recipient's cochlea, the likely position of the most basal electrode is estimated (or basal electrode depth of insertion), the mean modiolar proximity of the middle electrodes (electrode ˜8 to electrode ˜15) is estimated and the likely position of the most apical electrode is estimated. The Monte Carlo process produces many likely paths or positions of the electrode array. Paths that are unlikely are filtered out. The remaining paths or positions are averaged to form a final estimate of the placement of the electrode array inside of the cochlear after the insertion of the array during surgery.

In certain examples, the accuracy of the positioning models can be improved by applying metrics or classifications of cochlea morphology from pre-operative imaging. The method would be to segment the pre-op imaging to create a 3D model and then apply selected metrics such as volume or cross-sectional area of the scala as additional inputs for machine-learning algorithms.

The techniques presented herein can be applied in a number of different manners. For example, the techniques presented herein can be used for monitoring pre-curved perimodiolar electrode array insertion. That is, with a detailed knowledge of the behavior of an electrode array when it is taking an optimal insertion, and in comparison to when it is taking a sub-optimal trajectory, it is possible to apply the above techniques to detect, in real-time (e.g., during the course of an electrode array insertion), when the electrode array is following a sub-optimal trajectory. This is done by forming an estimation of the shape of the electrode array in the cochlea relative to the modiolus and the cochlea duct.

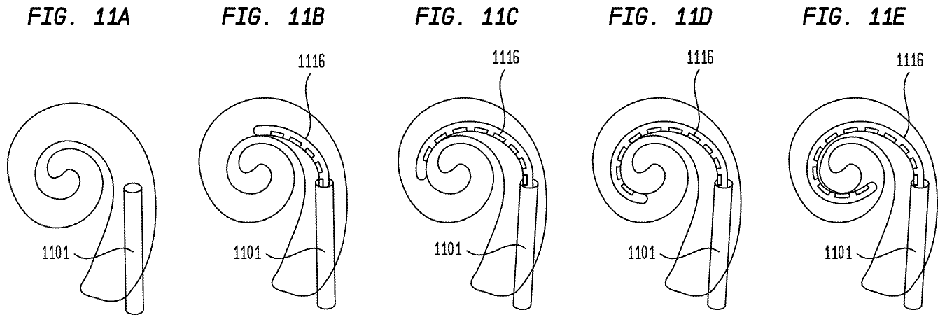

For example, with a perimodiolar electrode array, the electrodes should stay close to the modiolus during an optimal insertion (e.g., the electrode array moves smoothly around the modiolus, with the apical section of the electrode array maintaining close contact for the duration of the insertion). An example of this dynamic is depicted in FIGS. 11A-11E which show different stages in which an electrode array 1116 is inserted in a plastic model of a cochlea using a sheath 1101. FIGS. 11A-11E illustrate an electrode array trajectory in a relatively good insertion dynamic. After full deployment of the electrode array 1116, the sheath 1101 is removed.

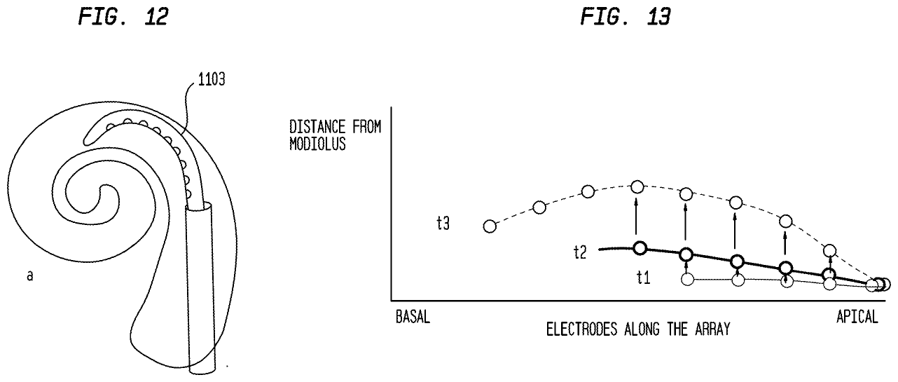

If the apical (distal) tip of the electrode array 1116 catches on a ridge of the modiolus during insertion, then the mid-section of the electrode array tends to arch out away from the modiolus and toward the lateral wall, as shown in FIG. 12 via the overlaid lines 1103. This dynamic can be identified using the modiolar proximity detection described above, making it possible to infer that the tip has become stuck. That is, if the estimated positioning of the electrode array indicates that the electrode array is close at the apical tip, but is starting to arch away from the modiolus further back from the tip, this is an indicator that the tip has become stuck on a ridge on the surface of the modiolus and continued insertion is pushing the electrode away from the modiolus. By detecting the situation in which the tip has become stuck, the system can indicate to the surgeon that the insertion is not going as intended so that the surgeon can take corrective action. The simplest action to correct this situation would be to withdraw the electrode slightly in an effort to release the tip from the ridge on which it is caught. The techniques presented herein also make it possible to detect when the tip of the electrode array is not against the modiolus, potentially indicating that the electrode array has been inserted backward or some other unexpected situation has occurred.

FIG. 13 is graph including curves indicating of the distance of the electrodes of an electrode array relative to the modiolus as the insertion progresses when the tip becomes stuck. Three times are shown. At t1, 5 electrodes are deployed from the sheath and the contacts outside the sheath are close to the modiolus. At t2, 6 electrodes are deployed, the tip is stuck on the modiolus, and proximal electrodes are further away from the modiolus. At t3, 9 electrodes are deployed, the tip is stuck on the modiolus, and proximal electrodes are yet further from the modiolus. This data could be provided to a surgeon or a robot performing robotic insertion of stimulating assemblies. In the case of a robot, a closed loop feedback system could be implemented to manage the optimal insertion of the electrode array, with the robot stopping insertion and indicating to the surgeon that a corrective action is needed. Alternatively, the robot could attempt an automated corrective action such as a slight withdrawal and repeat insertion.

In certain examples, the system can compare the position of the electrode array to known profiles of the shape of an electrode array, relative to the modiolus and cochlear duct in an optimal insertion. As such, the system could determine that the tip is likely stuck and then suggest corrective action. The profiles of the shape of an electrode array could include multiple measurements such as the estimate of position at different points in time and the change in position across multiple electrodes. There would also be potential to apply machine learning techniques to develop the model of electrode becoming stuck and failing to advance.

In certain embodiments, additional information that could be applied to enhance the algorithm, including measurement of insertion progress. If the surgeon is still pushing the electrode array forward while the distance of the electrodes from the modiolus is increasing, this would be consistent with a tip being caught and the electrode array arching out to the lateral wall. The insertion progress could be monitored by a number of means including: measuring electrode impedances as each contact exits the sheath; video observing the movement of the electrode array insertion from outside; video via an optical fiber which could be independent, or built into the electrode array or sheath; or intra-operative navigation equipment monitoring the movement of the electrode array on the outside of the cochlea.

As noted, it may be advantageous to incorporate these technique in a robot-controlled insertion system. This would provide a more stable electrode position throughout the insertion and facilitate a slow insertion speed, both of which would reduce “noise” from the dynamic and make it easier for the algorithm to confirm whether an electrode array is starting to arch outward. Feedback could also be incorporated to stop insertion as soon as the system suspects a tip that is caught so that corrective action could be taken. As noted, a corrective action could be as simple as reversing the insertion for a short distance which would allow the electrode array to settle back into the intended position prior to restarting the insertion. An alternative correction could be by means of adjusting the angle of the tip of the electrode by various means described below.

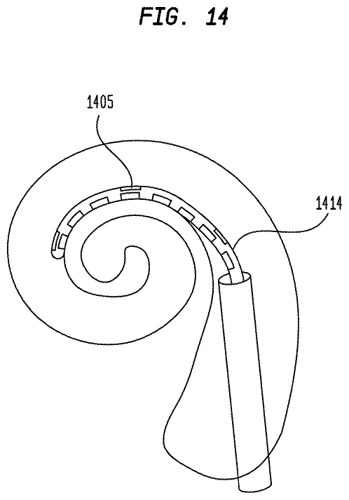

The examples of FIGS. 11A-11E, 12, and 13 have been described as using the stimulating electrodes on the electrode array, which are on the modiolar side of the electrode array. It is also possible to incorporate electrodes on the lateral surface of the electrode array to allow detection of position of the electrode array relative to the lateral wall (e.g., enable addition of a measure of proximity to the lateral wall. These electrodes could be small in area as they are only for detection of position, not for stimulation. FIG. 14 is a diagram illustrating an electrode array 1414 having lateral electrodes 1405.

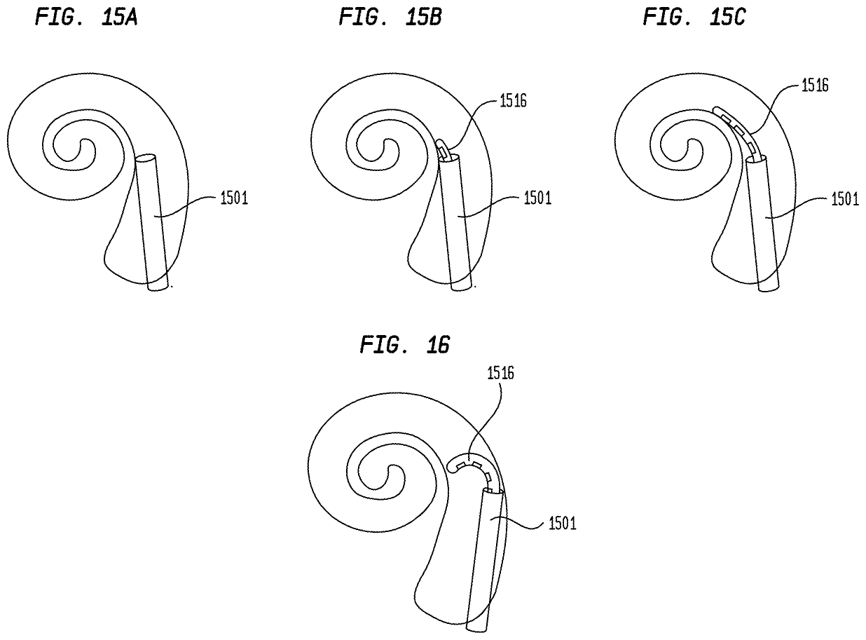

As noted, pre-curved stimulating assemblies are generally inserted using a stiffening sheath that maintains the electrode array in a straight arrangement prior to insertion, then is removed during insertion. The position of the sheath in the cochlea can be important to ensuring a smooth insertion. Ideally, the tip of the sheath is positioned close to the modiolus so that the electrode array runs smoothly onto the modiolar wall as it exits the sheath. FIGS. 15A-15C illustrative correct positioning of a sheath 1501 during insertion of an electrode array 1516. However, as shown in FIG. 16, if the sheath 1501 is held with the tip toward the lateral wall, then in the case of a wide lumen, the electrode array 1516 can bend over immediately on exiting the sheath 1501 and catch on the modiolus close to the sheath tip.

A solution to this issue is to include electrodes on the sheath and apply the same measurement technique as described above to determine the distance of the sheath from the modiolus and/or lateral wall. That is, by incorporating electrodes on the sheath it is possible to estimate the position of the sheath in the cochlea relative to the modiolus. If the tip is not sufficiently close to the modiolus, this could indicate that the sheath is in a sub-optimal angle or depth and hence pose a risk of complications during insertion. A system could monitor the position of the sheath relative to the modiolus and lateral wall (and thus the position in the cochlear duct) and indicate to the surgeon whether the sheath is being held at an optimal angle and depth. Note that the sheath is intended to be held in a static position during deployment of the electrode array, however it is known that sometimes surgeons unintentionally change the angle or depth of the sheath during deployment of the electrode. A system could continuously monitor the position of the sheath and alert the surgeon of an unintended change. These feedback signals could be provided to a surgeon or a robot performing robotic insertion of electrode array. In the case of a robot, a closed loop feedback system could be implemented to manage the optimal positioning of the sheath and insertion of the electrode.

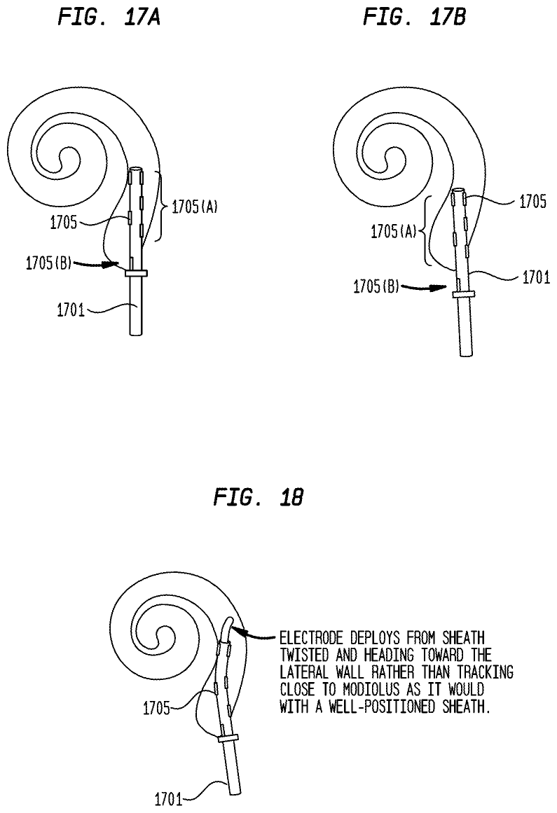

FIGS. 17A and 17B illustrate a sheath 1701 with electrodes 1705 disposed therein. FIG. 17A illustrates the sheath 1701 in an optimal/preferred position, while FIG. 17B illustrates the sheath 1701 not fully inserted. The arrangement of FIG. 17B could be detected and signaled to the surgeon (or a robot) to adjust the angle and depth of the sheath to optimize the insertion before or while advancing the electrode array out of the sheath 1701. In one example, the impedances of the electrodes 1705(A) can be used to determine if the tip of the sheath 1701 is in the optimal position close to the modiolus, while the impedance of electrode 1705(B) can be used to confirm that the sheath 1701 is fully inserted with the stopper resting on the cochlea. If the sheath 1701 is accidentally held outside the cochlea as shown in FIG. 17B, then electrode 1705(B) would lose connection with the cochlea fluid and go to high impedance.

It is noted that it is possible to measure sheath location with electrodes on one side of the sheath. However, it is also advantageous to include electrodes on either side (medial and lateral sides) of the sheath as shown in FIGS. 17A and 17B to improve the accuracy of estimate of the relative position within the cochlea duct since the algorithm detects the volume of the space immediately adjacent to the electrode and thus can determine the relative distance to the nearest wall with greater accuracy.

In certain examples, a sheath is held too hard against the modiolus, which can bend or kink the sheath. This can jam the electrode array in the sheath preventing insertion, as shown in FIG. 18. A bend in the sheath can also cause the electrode array to twist and exit the sheath in a trajectory toward the lateral wall and potentially impacting on the lateral wall. Using the techniques presented herein, the measurement of the distance from the modiolus and lateral wall can be used to detect that the electrode array is not in the expected position as it deploys from the sheath.

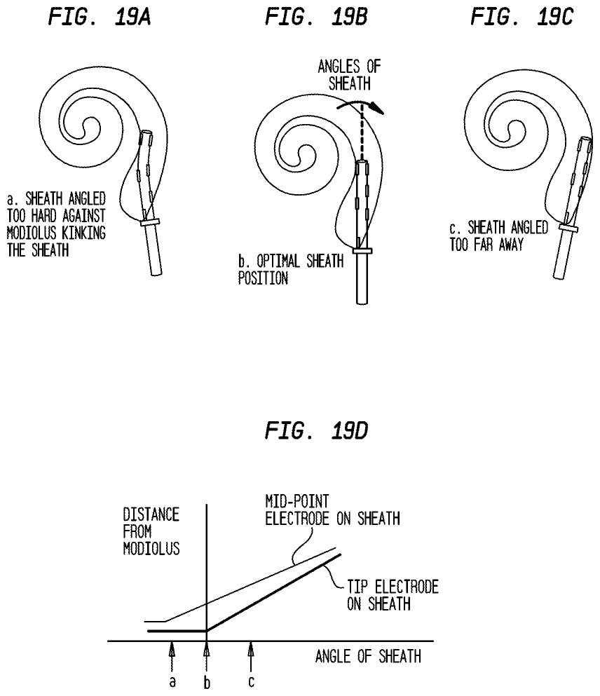

To determine if the sheath is in an optimal position, an algorithm may be applied based on the model depicted in FIGS. 19A-19D. FIGS. 19A, 19B, and 19C illustrate three positions of sheath when inserted into the cochlea via the round window. In operation, the surgeon can move the angle of the sheath from low to high and back again to traverse the space. The measurement system can then record the spacing of the tip and mid-point electrode to the modiolus and estimate the optimal angle where the tip electrode has just reached close proximity to the modiolus, and the mid-point electrode is spaced by some reasonable distance. An algorithm based on the curves shown in FIG. 19D can be used to determine whether the angle of the sheath has been optimized.

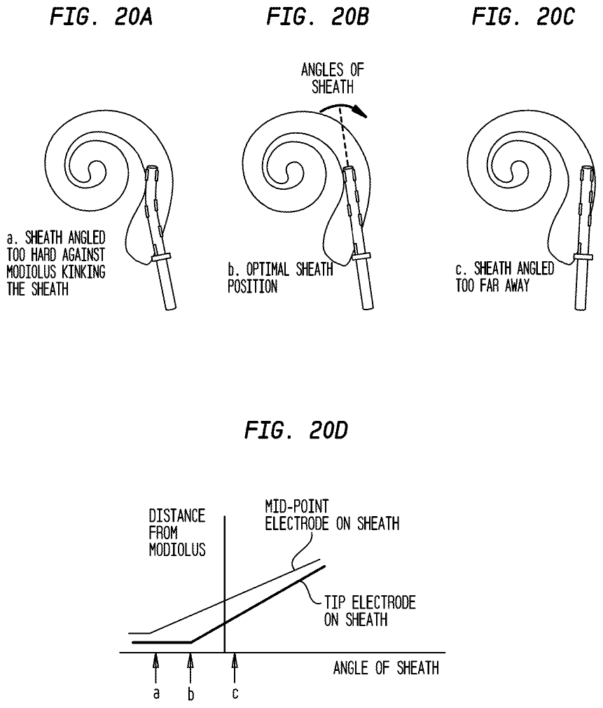

The model of the curves shown in FIGS. 19A-19D can also be applied for electrodes inserted through a cochleostomy. If inserted through a cochleostomy, the angles for the three cases would be modified by an amount depending on the exact position of the cochleostomy as shown in FIGS. 20A-20D. In general, the absolute angle of each of the three positions shown in FIGS. 20A, 20B, and 20C would be further anticlockwise relative to the orientation of the cochlea, however the difference in angle between FIGS. 20A, 20B, and 20C would be similar for cochleostomy to that of round window insertion. Again, an algorithm based on the curves shown in FIG. 20D can be used to determine whether the angle of the sheath has been optimized.

As noted, FIGS. 17A-20D illustrate concepts using electrodes attached to the sheath. It is to be appreciated that number of implementations are possible with this arrangement. For example, in certain embodiments, electrodes on the sheath may be connected back to the measurement system by wires or the electrodes could be connected to a small wireless transmitter incorporated in the sheath to transmit the information back without the inconvenience of a wired connection. In certain embodiments, the electrodes on the sheath could be connected to the electrodes of the intracochlear electrode array by means of brushes on the inside of the sheath to connect to the contacts of the electrode array. Moreover, instead of incorporating electrodes on the sheath, the contacts of the electrode array could be utilized simply by creating an opening in the sheath over a number of the contacts. Yet a further refinement of the above is to add a strain gauge to the sheath to detect whether it has been deformed. The signal from the strain gauge can be included with analysis of the position of the electrode array to further improve the potential to detect sub-optimal sheath position.

It is to be appreciated that straight (e.g., not pre-curved) electrode array insertions can be monitored in a similar manner to that described for perimodiolar electrode array insertions. The characteristic trajectory of a straight electrode array is known. By applying a detailed knowledge of the optimal insertion trajectory with known sub-optimal trajectories, it is possible to determine when the electrode array is deviating from the optimal insertion trajectory.

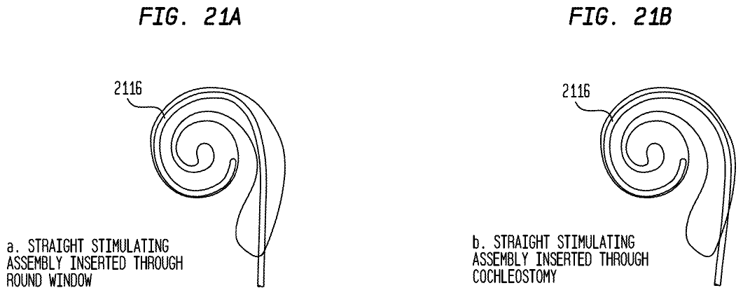

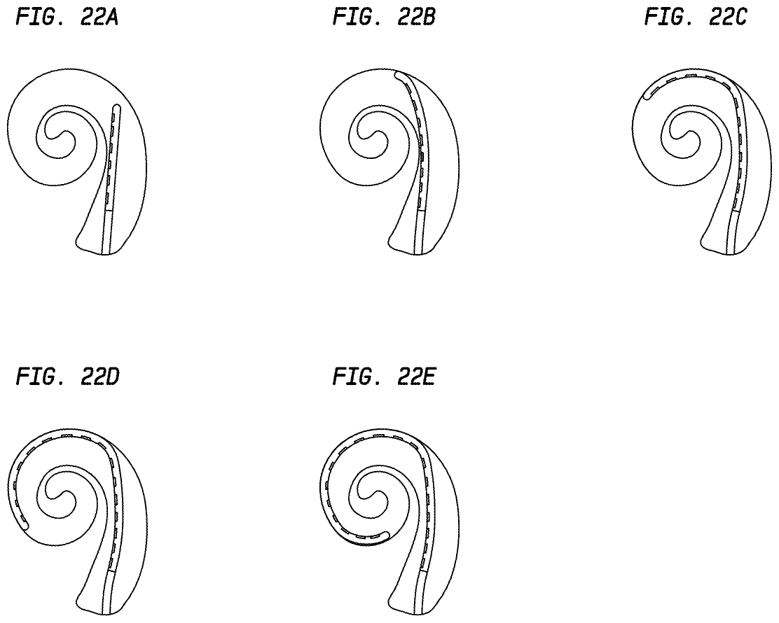

More specifically, when a straight electrode array is being inserted in an ideal manner, the electrode array moves smoothly around the lateral wall. The trajectory and position of the electrode array relative to the cochlea duct depends on whether the electrode is inserted through a round window or cochleostomy. FIG. 21A depict a good insertions for an electrode array 2116 via the round window and a cochleostomy, respectively. This contour of the electrode array 2116 in the final position as shown is also reasonably descriptive of the trajectory followed by the array during an optimal insertion. As shown in FIG. 21A, when inserted through the round window, the electrode array 2116 glances past the modiolus before heading out to the lateral wall and staying on the lateral wall up to the most-apical point of insertion. As shown in FIG. 21B, when inserted through a cochleostomy, the electrode array 2116 travels around the lateral wall of the duct. FIGS. 22A-22E illustrate an example of this dynamic, which shows the trajectory of the electrode array 2116 in a number of stages of insertion in a plastic cochlea model.

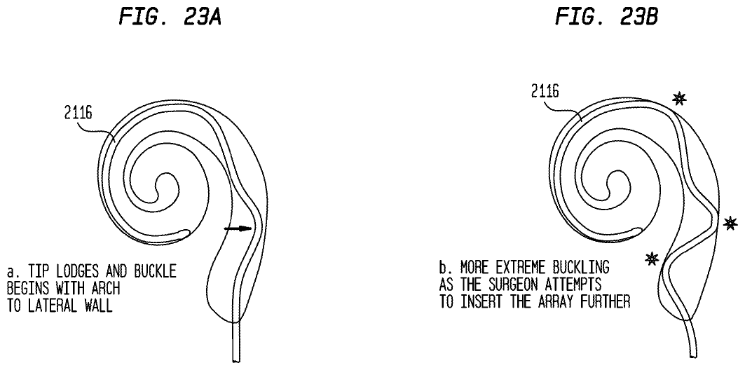

There are a number of ways in which an insertion of a straight electrode array can deviate from the optimal trajectory. These cases can be detected during insertion by comparing the position of the electrode array as it is inserted with the optimal insertions of FIGS. 21A and 21B. The techniques described below can be used with both round window and cochleostomy insertions. The round window insertion is used for examples below with reference to FIGS. 23A, 23B, 24A, 24B, 25A, 25B, 26A, and 26B.

More specifically, FIGS. 23A and 23B illustrate a dynamic where the electrode array 2116 begins to buckle in the basal section as the tip of the electrode array lodges in the tissue of the lateral wall. That is, the basal section of the array begins to buckle and arch out toward the lateral wall (as shown by the arrow in FIG. 23A). With the tip lodged and not moving further inward, continued insertion by the surgeon results in more extreme buckling in the basal section (as shown by the arrows in FIG. 13B) of the electrode array which leads to undesirable pressure points on the lateral and modiolar tissues. Such pressure points can cause trauma to a number of structures including: the stria vascularis (potentially causing a bleed and/or affecting blood supply); or the ossia spiral lamina (potentially causing a fracture and bleeding). These can lead to loss of residual hearing and growth of fibrous tissue which are best to be avoided.

This basal-buckling dynamic can be identified during an insertion by the modiolar proximity detector algorithm at an early stage as depicted in FIG. 23A, allowing the system to alert the surgeon to the fact that the electrode is beginning to buckle instead of advancing into the cochlea. The surgeons can therefore stop insertion to avoid the extreme buckling and consider corrective actions.

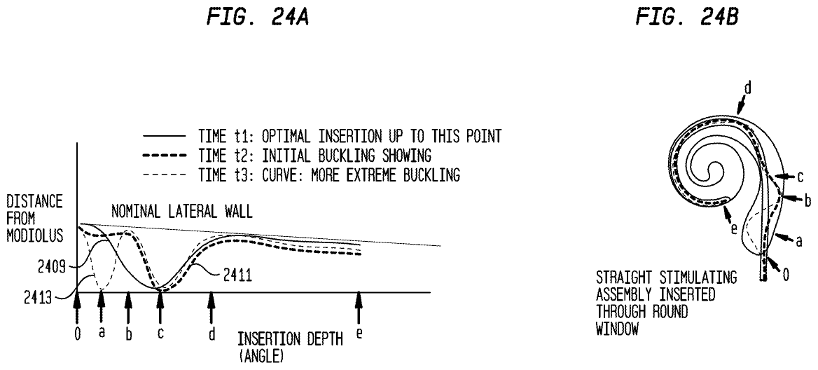

FIGS. 24A and 24B demonstrate a more detailed method by which this situation can be detected based on graphs of the distance from the modiolus (and lateral wall) for an electrode array at three points in time during insertion. In particular at time t1, the solid curve 2409 shown in FIG. 24A depicts the position of the electrode array at the point in the insertion when the tip of the electrode array has just lodged in the lateral wall at angular depth “e” which will prevent it from moving further into the cochlea. Up to this point in time, the electrode array has adopted an optimal trajectory and position in the cochlea. Curve 2409 is a baseline against which later time points can be compared.

At time t2, dashed curve 2411 depicts a point later in time when the electrode array has been inserted further. However, since the tip is lodged at location “e,” a buckle has begun in the basal section of the electrode array. Note that the basal position of the electrode array has morphed from the curve 2409 to the curve 2411 as the electrode array arches out to the lateral wall at angular depth “b”.

At time t3, the dotted curve 2413 depicts a point later again when the electrode array has been inserted yet further and a more severe buckle has appeared. Again, the tip has not advanced further into the cochlea, but rather the basal position has further morphed from the curve 2411 to the curve 2413 causing the electrode to arch toward the modiolar wall at angular depth “a” and toward the lateral wall at angular depth “b”. In FIG. 24B, the curves 2409, 2411, and 2413 are shown offset from one another merely for ease of illustration.

The change in position of the electrode array in the cochlea can be measured using the modiolar proximity algorithm and a determination made that the electrode array has begun to buckle based on the difference in profile of the electrode of the purple and red curves in comparison to the grey curve. The difference between the curves can be determined by identifying the notable points where the electrode is closest to the modiolar or lateral walls i.e., angular locations a, b, c, and d. In general, FIG. 24A depicts the distance from modiolus for insertion through the round window comparing optimal insertion at time t1 when the tip lodges in the lateral wall, time t2 when initial buckling occurs, and time t3 when more extreme buckling has occurred. FIG. 24B shows the position of the electrode at the three time points overlaid on the cochlea model

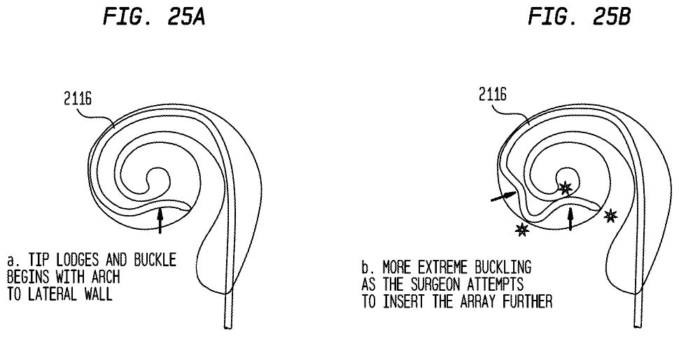

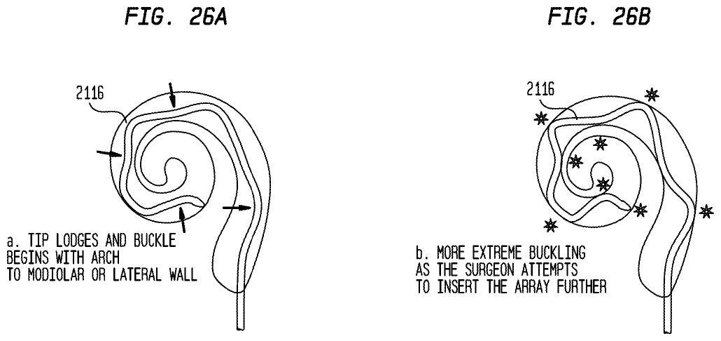

Some other potential buckling scenarios are shown FIGS. 25A-25B and 26A-26B. In particular, FIGS. 25A and 25B illustrate a dynamic wherein the electrode array begins to buckle in the apical section as the tip of the electrode array lodges in the tissue of the lateral wall. In this example, initially the electrode array begins to buckle in the apical section rather than move further into the cochlea. The buckle is indicated by the arrow in FIG. 25B where the electrode array arches toward the modiolar wall. If the surgeon continues to push the electrode array into the cochlea, the buckling in the apical section becomes more extreme as indicated by the arrows in FIG. 25B. This more extreme buckling can cause high pressure points on the lateral wall at the points indicated by the stars in FIG. 25B. Such pressure points can cause trauma to delicate structures in the cochlea and are best to be avoided.

FIGS. 26A and 26B describe yet another dynamic wherein the electrode array begins to buckle in multiple locations along the array as the tip of the electrode array lodges in the tissue of the lateral wall. In this example, initially the electrode array begins to buckle in the multiple sections rather than move further into the cochlea. The buckle is indicated by the arrows where the electrode array buckles in toward the modiolar wall or outward to the lateral wall. If the surgeon continues to attempt to push the electrode array into the cochlea, the buckling in the apical section becomes more extreme as indicated by the arrows in FIG. 26A. This more extreme buckling can cause high pressure points on the lateral wall at the points indicated by the stars in FIG. 26B. Such pressure points can cause trauma to delicate structures in the cochlea and are best to be avoided.

Both of the scenarios of FIGS. 25A-25B and 26A-26B can be detected by the modiolar proximity detector algorithm using the techniques described for the basal buckling scenario.

Buckling of straight stimulating assemblies depicted above cannot be detected by feel since the stimulating assemblies are too flexible to present any sensation back to the surgeon. Thus, an objective measure to identify this buckling meets a currently unmet need. The exact position of the buckling in the electrode array may vary depending on the exact dimensions of the cochlea and the characteristics of the electrode array. The variation in the dynamic of a given electrode array can be explored in models and temporal bones so that a more specific model of buckling can be determined for a given electrode design.

It is also possible to develop an atlas of electrode behaviors from post-operative and/or intraoperative imaging in clinical subjects. Such an approach may be the foundation of a database of cases which could be used with Machine Learning or AI techniques to refine a detection algorithm. In addition, one potential corrective action on detection of a straight electrode array beginning to buckle is to slightly withdraw the electrode array to achieve an electrode array position as close as possible to an optimal position. This can be confirmed by comparison with the optimal profile for a straight electrode array.

A further refinement is to combine the concepts above with a model of the anatomy based on pre-operative imaging and planning. This would involve taking pre-operative images and segmenting the anatomy to create a three dimensional model of the anatomy. The sheath can then be superimposed on the model in a planning step to determine optimal angle of the sheath relative to the anatomy and estimates of the distance from the modiolus of the electrodes on the sheath that would match an optimal sheath angle. The pre-operative plan with optimal angle of the sheath might also be used in surgery with intraoperative navigation which could also be used to compare and confirm the position of the sheath indicated by the modiolar proximity measurements, providing an even more robust algorithm to confirm optimal sheath position. Application of metrics or classifications of morphology of the cochlea would also allow higher accuracy of the estimate of misplacement of electrodes.

As noted above, in certain embodiments, information associated with an electrode array position can be provided to a recipient. This information can be provided as visible feedback (e.g., information provided via, for example, a display screen, etc.), audible feedback (e.g., audio signals played via a speaker, etc.) tactile information, data provided to a control system (e.g., in robotic insertion), etc. That is, the information associated with an electrode array position can be determined and output in a number of different manners.

In certain aspects of the techniques presented herein, the feedback can include information relating to a certainty of electrode array position during insertion. As noted above, certain positioning models presented herein use probabilistic or statistical approaches to generate an estimate/prediction of the position of one or more parts of an electrode array. A number of probabilistic or statistical techniques can provide a measure of the accuracy or certainty of such predictions/estimations. In accordance with certain embodiments presented herein, this degree certainty or likely accuracy of that estimate can be conveyed to a user, such as to a surgeon while the electrode array is being placed, to a clinician (e.g., audiologist) at some later date, etc.

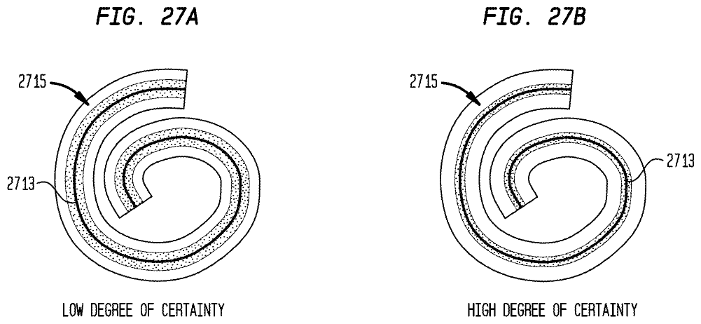

FIGS. 27A and 27B illustrate one example technique to visually display the degree certainty or likely accuracy of an estimated position of an electrode array within a cochlea. More specifically, FIGS. 27A and 27B are example images that could be displayed to a user to indicate the degree of certainty of the electrode array. The images of FIGS. 27A and 27B could be displayed in the visual field of the operating microscope during surgery for example or could be displayed on any device with a suitable display screen.

In these drawings, the solid line 2713 represents the most likely location of the electrode array and the “fuzzy” line surrounding it indicates the degree of uncertainty, where a wider “fuzziness” equates to greater uncertainty. As such, FIG. 27A illustrates a highly uncertain estimate and FIG. 27B indicates a more certain estimate. The background in these figures indicates the likely location and shape of the cochlea duct into which the electrode array is placed. These could be determined from pre-operative imaging or from past databases of other cochleae shapes and sizes or some mixture of both. Note that there may be some uncertainty in the known width and shape of the cochlear duct and (although not shown in FIGS. 27A and 27B) this can be indicated by fuzziness in the yellow area. FIGS. 27A and 27B also show the situation for a fully inserted electrode array, but similar concepts can be used for an intraoperative display where the image would show the estimated depth of insertion and the display would change as the electrode array is advance into the cochlea as the insertion progresses.

It is also noted that (although not shown in FIGS. 27A and 27B), the degree of uncertainty may vary along the length of the array. It may also be useful to change the color of the line to highlight to the user (e.g., surgeon) which parts of the electrode array are more certain than others. Alternatively, it may be useful to use color to indicate to the surgeon which parts of the electrode array are inserted more optimally than others. For example, if the goal of a particular electrode array is to be inserted close to the modiolus of the cochlea, then those parts of the electrode array that are highly likely to be close to the modiolus could be colored/shaded in one manner whereas those parts of the electrode array that are highly likely to be distal to the modiolus could be colored/shaded in another manner. In between the display could present a color/shading that is a mixture, with a higher percentage of one color/shading for high certainty of a proximal placement and a higher percentage of another color/shading for a higher percentage of a more distal location. That is color, shading, etc. could be used as a visual indicator of certainty or of uncertainty.

In addition, contrast between the electrode color/shading and the background (cochlear duct) color/shading could also be used in the same way. More highly contrasting areas could indicate a high degree of certainly and less contrasting areas could indicate a low degree of certainty for example.

In further embodiments, the certainty of an estimated location could be provided as a numerical value from the probabilistic or statistical modelling process used to determine the position of one or more parts of the electrode array. This value could be displayed, for example, as a percentage probability in a window in the operating field of the microscope. The certainly could be displayed as a single value or as multiple values to represent multiple points along the electrode array. The numerical value(s) could also be combined with a pictorial representation of the electrode array, as described above, so numbers could be added to the pictogram above to indicate degree of certainty at a particular location along the electrode array. Note that numerical representations of assessed “goodness” of electrode array position as described above (e.g., close to modiolus scores a higher percentage than more distal) can also be provided as feedback to a user in the same was as certainty. In certain examples, numerical values for goodness of placement may be a better use of numerical feedback to a user than degree of certainty, which may be better represented visually as described in the examples of FIGS. 27A and 27B.

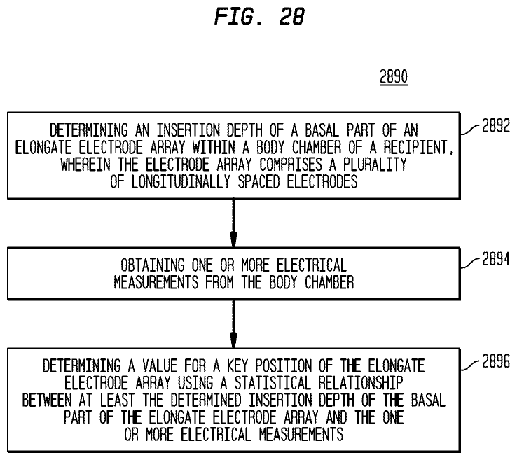

FIG. 28 is a flowchart of a method 2890, in accordance with certain embodiments presented herein. Method 2890 begins at 2892 where a system determines an insertion depth of a basal part of an elongate electrode array within a body chamber (e.g., cochlea) of a recipient, wherein the electrode array comprises a plurality of longitudinally spaced electrodes. At 2894, the system obtains one or more electrical measurements from the body chamber. At 2896, the system determines a value for a key position of the elongate electrode array using a statistical relationship between at least the determined insertion depth of the basal part of the elongate electrode array and the one or more electrical measurements.

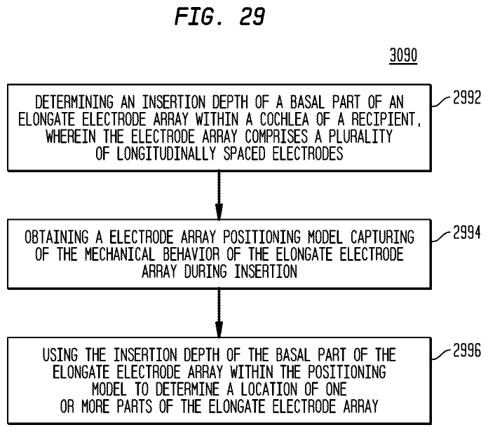

FIG. 29 is a flowchart of a method 2990, in accordance with certain embodiments presented herein. Method 2990 begins at 2992 where a system determines an insertion depth of a basal part of an elongate electrode array within a cochlea of a recipient, wherein the electrode array comprises a plurality of longitudinally spaced electrodes. At 2994, the system obtains a positioning model of the mechanical behavior of the elongate electrode array during insertion and with respect to proximity to the wall of the cochlea. At 2996, the systems uses the insertion depth of a basal part of an elongate electrode array within the model to determine an intra-surgical electrode location of one or more parts of the elongate electrode array.

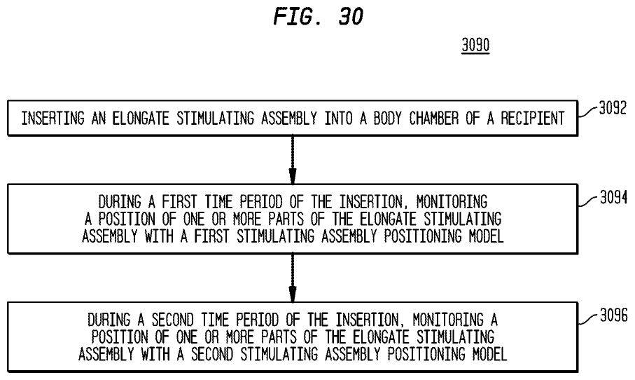

FIG. 30 is a flowchart of a method 3090, in accordance with certain embodiments presented herein. Method 3090 begins at 3092 where an elongate electrode array is inserted into a body chamber (e.g., cochlea) of a recipient. At 3094, during a first time period of the insertion, a system monitors a position of one or more parts of the elongate electrode array with a first positioning model. At 3096, during a second time period of the insertion, the system monitors a position of one or more parts of the elongate electrode array with a second positioning model.

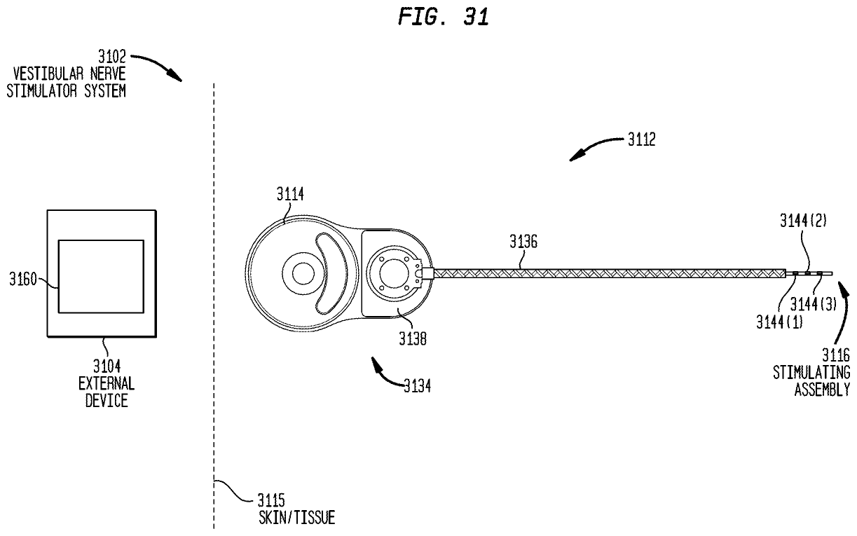

As previously described, the technology disclosed herein can be applied in any of a variety of circumstances and with a variety of different devices. Example devices that can benefit from technology disclosed herein are described in more detail in FIG. 31, below, which illustrates a vestibular stimulator. As noted elsewhere herein, the techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 31 illustrates an example vestibular stimulator system 3102, with which embodiments presented herein can be implemented. As shown, the vestibular stimulator system 3102 comprises an implantable component (vestibular stimulator) 3112 and an external device/component 3104 (e.g., external processing device, battery charger, remote control, etc.). The external device 3104 comprises a transceiver unit 3160. As such, the external device 3104 is configured to transfer data (and potentially power) to the vestibular stimulator 3112.

The vestibular stimulator 3112 comprises an implant body (main module) 3134, a lead region 3136, and an electrode array 3116, all configured to be implanted under the skin/tissue (tissue) 3115 of the recipient. The implant body 3134 generally comprises a hermetically-sealed housing 3138 in which RF interface circuitry, one or more rechargeable batteries, one or more processors, and a stimulator unit are disposed. The implant body 134 also includes an internal/implantable coil 3114 that is generally external to the housing 3138, but which is connected to the transceiver via a hermetic feedthrough (not shown).

The electrode array 3116 comprises a plurality of electrodes 3144(1)-(3) disposed in a carrier member (e.g., a flexible silicone body). In this specific example, the electrode array 3116 comprises three (3) stimulation electrodes, referred to as stimulation electrodes 3144(1), 3144(2), and 3144(3). The stimulation electrodes 3144(1), 3144(2), and 3144(3) function as an electrical interface for delivery of electrical stimulation signals to the recipient's vestibular system.

The electrode array 3116 is configured such that a surgeon can implant the electrode array adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

In operation, the vestibular stimulator 3112, the external device 3104, and/or another external device, can be configured to implement the techniques presented herein. That is, the vestibular stimulator 3112, possibly in combination with the external device 3104 and/or another external device, can include an evoked biological response analysis system, as described elsewhere herein.

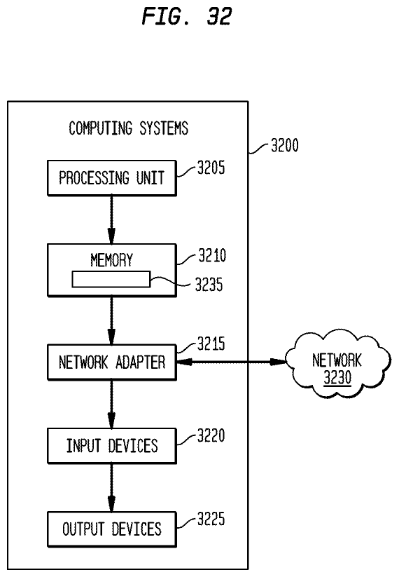

FIG. 32 illustrates an example of a suitable computing system 3200 with which one or more of the disclosed examples can be implemented. Computing systems, environments, or configurations that can be suitable for use with examples described herein include, but are not limited to, personal computers, server computers, hand-held devices, laptop devices, multiprocessor systems, microprocessor-based systems, programmable consumer electronics (e.g., smart phones), network PCs, minicomputers, mainframe computers, tablets, distributed computing environments that include any of the above systems or devices, and the like. The computing system 3200 can be a single virtual or physical device operating in a networked environment over communication links to one or more remote devices. The remote device can be an auditory prosthesis (e.g., an auditory prosthesis), a personal computer, a server, a router, a network personal computer, a peer device or other common network node.

In its most basic configuration, computing system 3200 includes at least one processing unit 3205 and memory 3210. The processing unit 3205 includes one or more hardware or software processors (e.g., Central Processing Units) that can obtain and execute instructions. The processing unit 3205 can communicate with and control the performance of other components of the computing system 3200.

The memory 3210 is one or more software or hardware-based computer-readable storage media operable to store information accessible by the processing unit 3205. The memory 3210 can store, among other things, instructions executable by the processing unit 3205 to implement applications or cause performance of operations described herein, as well as other data. The memory 3210 can be volatile memory (e.g., RAM), non-volatile memory (e.g., ROM), or combinations thereof. The memory 3210 can include transitory memory or non-transitory memory. The memory 3210 can also include one or more removable or non-removable storage devices. In examples, the memory 3210 can include RAM, ROM, EEPROM (Electronically-Erasable Programmable Read-Only Memory), flash memory, optical disc storage, magnetic storage, solid state storage, or any other memory media usable to store information for later access. In examples, the memory 3210 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 3210 can include wired media such as a wired network or direct-wired connection, and wireless media such as acoustic, RF, infrared and other wireless media, or combinations thereof. In certain embodiments, the memory 3210 comprises electrode array positioning logic 3235 that, when executed, enables the processing unit 3205 to perform aspects of the techniques presented.

In the illustrated example, the system 3200 further includes a network adapter 3215, one or more input devices 3220, and one or more output devices 3225. The system 3200 can include other components, such as a system bus, component interfaces, a graphics system, a power source (e.g., a battery), among other components.

The network adapter 3215 is a component of the computing system 3200 that provides network access (e.g., access to at least one network 3230). The network adapter 3215 can provide wired or wireless network access and can support one or more of a variety of communication technologies and protocols, such as ETHERNET, cellular, BLUETOOTH, near-field communication, and RF (Radiofrequency), among others. The network adapter 3215 can include one or more antennas and associated components configured for wireless communication according to one or more wireless communication technologies and protocols.

The one or more input devices 3220 are devices over which the computing system 3200 receives input from a user. The one or more input devices 3220 can include physically-actuatable user-interface elements (e.g., buttons, switches, or dials), touch screens, keyboards, mice, pens, and voice input devices, among others input devices.

The one or more output devices 3225 are devices by which the computing system 3200 is able to provide output to a user. The output devices 3225 can include, displays, speakers, and printers, among other output devices.

It is to be appreciated that the arrangement for computing system 3200 shown in FIG. 32 is merely illustrative and that aspects of the techniques presented herein may be implemented at a number of different types of systems/devices. For example, the computing system 3200 could be a laptop computer, tablet computer, mobile phone, surgical system, etc.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. A method, comprising:
determining an insertion depth of a basal part of an elongate electrode array within a body chamber of a recipient, wherein the electrode array comprises a plurality of longitudinally spaced electrodes;
obtaining one or more electrical measurements from the body chamber of the recipient; and
determining a value for a key position of the elongate electrode array using a relationship between at least the determined insertion depth of the basal part of the elongate electrode array and the one or more electrical measurements.

2. The method of claim 1, wherein the relationship is mathematically described by at least one of a statistical model or a probabilistic model.

3. The method of claim 1, wherein determining a value for a key position of the elongate electrode array comprises:
determining a distance between one or more of the plurality of longitudinally spaced electrodes and an adjacent wall of the body chamber.

4. The method of claim 1, wherein determining a value for a key position of the elongate electrode array comprises:
determining an angular insertion depth of the elongate electrode array.

5. The method of claim 1, wherein determining a value for a key position of the elongate electrode array comprises:
determining polar or Cartesian coordinates of one or more of the plurality of longitudinally spaced electrodes.

6. The method of claim 1, wherein determining a value for a key position of the elongate electrode array comprises:
determining a value for a non-basal area of the elongate electrode array.

7. The method of claim 1, wherein determining a value for a key position of the elongate electrode array comprises:
determining a value for a point near middle of the elongate electrode array.

8. The method of claim 7, wherein the value for a point near middle of the elongate electrode array is a modiolar proximity of the point near middle of the elongate electrode array.

9. The method of claim 1, wherein determining the insertion depth of the basal part of the elongate electrode array comprises:
determining an insertion depth of a most basal electrode.

10. The method of claim 1, wherein determining the insertion depth of the basal part of the elongate electrode array comprises:
determining the insertion depth of the basal part of the elongate electrode array based on one or more electrical measurements obtained from the elongate electrode array.

11. The method of claim 1, wherein determining the insertion depth of the basal part of the elongate electrode array comprises:
determining the insertion depth of the basal part of the elongate electrode array based on visual markers on the elongate electrode array.

12. The method of claim 1, wherein the elongate electrode array is inserted via robotic insertion, and wherein determining the insertion depth of the basal part of the elongate electrode array comprises:
determining the insertion depth based on data captured during the robotic insertion of the elongate electrode array.

13. The method of claim 1, wherein obtaining one or more electrical measurements from the body chamber comprises:
obtaining a plurality of impedance measurements from the elongate electrode array.

14. The method of claim 13, wherein obtaining a plurality of impedance measurements comprises:
obtaining transimpedance measurements from the elongate electrode array.

15. The method of claim 1, wherein the electrode array is inserted via stiffening sheath including one or more electrodes, and wherein obtaining one or more electrical measurements from the body chamber comprises:
obtaining the one or more electrical measurements using at least the one or more electrodes of the stiffening sheath.

16. The method of claim 1, wherein the electrode array comprising a plurality of medial electrodes and a plurality of lateral electrodes, and wherein obtaining one or more electrical measurements from the body chamber comprises:
obtaining the one or more electrical measurements using at least the plurality of lateral electrodes.

17. The method of claim 1, wherein determining a value for a key position of the elongate electrode array using a statistical relationship between at least the determined insertion depth of the basal part of the elongate electrode array and the one or more electrical measurements further comprises:
generating a plurality of estimates of the value of the position of the elongate electrode array using a probabilistic model of electrode array insertions; and
determining a final value for the key position of the elongate electrode array from the plurality of estimates.

18. The method of claim 17, wherein determining the final value for the key position of the elongate electrode array from the plurality of estimates comprises:
determining an average of the plurality of estimates.

19. The method of claim 17, wherein determining the final value for the key position of the elongate electrode array from the plurality of estimates comprises:
filtering the plurality of estimates based on determined physical dimensions of the body chamber.

20. The method of claim 1, wherein determining a value for a key position of the elongate electrode array using a statistical relationship between at least the determined insertion depth of the basal part of the elongate electrode array and the one or more electrical measurements further comprises:
generating a plurality of estimates of the key value using a first principles model of electrode array insertions.

21-32. (canceled)

33. A method, comprising:
inserting an elongate electrode array into a body chamber of a recipient;
during a first time period of the insertion, monitoring a position of one or more parts of the elongate electrode array with a first positioning model; and
during a second time period of the insertion, monitoring a position of one or more parts of the elongate electrode array with a second positioning model.

34. The method of claim 33, wherein the first positioning model is a first principles model derived from mechanical properties of the electrode array.

35. The method of claim 33, wherein the second positioning model is a probabilistic model configured to generate a plurality of estimates of the position of one or more parts of the elongate electrode array at a given point in time.

36. The method of claim 35, wherein the probabilistic model uses at least one of a Monte Carlo technique, Markov Monte Carlo technique, a Hidden Markov Model technique, or a Bayesian inference technique to generate the plurality of estimates of the position of one or more parts of the elongate electrode array.

37. One or more non-transitory computer readable storage media comprising instructions that, when executed by a processor, cause the processor to:
determine an insertion depth of a basal part of an elongate electrode array within a body chamber of a recipient;
receive one or more electrical measurements obtained from the body chamber of the recipient; and
estimate a position of the elongate electrode array using a statistical or probabilistic relationship between at least the determined insertion depth of the basal part of the elongate electrode array and the one or more electrical measurements.

38. The one or more non-transitory computer readable storage media of claim 37, wherein the electrode array comprises a plurality of longitudinally spaced electrodes, and wherein the instructions that, when executed, cause the processor to estimate a position of the elongate electrode array comprise instructions that, when executed, cause the processor to:
estimate a distance between one or more of the plurality of longitudinally spaced electrodes and an adjacent wall of the body chamber.

39. The one or more non-transitory computer readable storage media of claim 37, wherein the instructions that, when executed, cause the processor to estimate a position of the elongate electrode array comprise instructions that, when executed, cause the processor to:
estimate an angular insertion depth of the elongate electrode array.

40. The one or more non-transitory computer readable storage media of claim 37, wherein the instructions that, when executed, cause the processor to estimate a position of the elongate electrode array comprise instructions that, when executed, cause the processor to:
estimate polar or Cartesian coordinates of one or more electrodes of the electrode array.

41. The one or more non-transitory computer readable storage media of claim 37, wherein the instructions that, when executed, cause the processor to estimate a position of the elongate electrode array comprise instructions that, when executed, cause the processor to:
estimate a position of a non-basal area of the elongate electrode array.

42. The one or more non-transitory computer readable storage media of claim 37, wherein the instructions that, when executed, cause the processor to estimate a position of the elongate electrode array comprise instructions that, when executed, cause the processor to:
estimate a position of a point near middle of the elongate electrode array.

43. The one or more non-transitory computer readable storage media of claim 42, wherein the position for a point near middle of the elongate electrode array is a modiolar proximity of the point near middle of the elongate electrode array.

44-47. (canceled)
